(19) United States
(12) Patent Application Publication
(10) Pub. No.: US 2007/0027486 A1
(43) Pub. Date: Feb. 1, 2007

(54) Medical devices for enhancing intrinsic neural activity

(75) Inventor: Randolph K. Armstrong, Houston, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/193,842

(22) Filed: Jul. 29, 2005

(51) Int. Cl. A61N 1/18

(52) U.S. Cl. 607/2; 607/45

(57) ABSTRACT

A method, system, and an apparatus are provided for providing an electrical neurostimulation therapy to a patient. The method comprises generating an electrical biasing signal defined by a plurality of parameters, at least one of which comprises a random value within a defined range, and applying the electrical biasing signal to a neural structure to bias an intrinsic neural signal on the structure. Neurostimulators and neurostimulation systems are provided for generating such a biasing signal and applying the signal to the neural structure, and include a stimulus generator for generating the signal, one or more electrodes for delivering the signal to a neural structure, and a controller for applying the signal to the electrodes.

### CROSS-REFERENCE TO RELATED APPLICATION

This application is a related application to U.S. patent application Ser. No. ______, entitled “Enhancing Intrinsic Neural Activity Using A Medical Device,” which is filed on the same date as the present application and in the name of the same inventors.

### BACKGROUND OF THE INVENTION

1. Field of the Invention

This invention relates generally to medical devices and, more particularly, to methods, apparatus, and systems for enhancing intrinsic neural activity in biological tissue to treat a medical condition of a patient.

2. Description of the Related Art

The human brain resides in the cranial cavity of the skull and controls the central nervous system (CNS) in a supervisory role. The central nervous system is generally a hub of a variety of electrical and/or neural activity requiring appropriate management. For example, properly controlled electrical or neural activity enables the human brain to manage various mental and body functions in a normal manner. However, abnormal electrical and/or neural activity is associated with different diseases and disorders in the central and peripheral nervous systems. In addition to a drug regimen or surgical intervention, potential treatments for such diseases and disorders include implantation of a medical device in a patient for electrical stimulation of body tissue. In particular, by selectively applying therapeutic electrical signals to one or more electrodes coupled to the patient's neural tissue, an implantable medical device may electrically stimulate a target neural tissue location. This stimulation may be used to treat a neurological disease, condition or disorder.

Therapeutic electrical signals may be used to stimulate cranial nerves such as the vagus nerve to generate afferent action potentials and thereby increase the flow of neural signals up the nerve, toward the brain. Therapeutic electrical signals may also be used to inhibit neural activity and to block neural impulses from moving up the nerve. Therapeutic electrical stimulation of the vagus nerve has been used to treat epilepsy and depression. Vagus nerve stimulation (VNS) therapy for treatment of epilepsy is described in many U.S. patents including U.S. Pat. Nos. 4,702,254, 4,867,164, and 5,025,807, which are incorporated herein by reference.

To provide vagus nerve stimulation to a patient, a neurostimulator device may be implanted in a target location in the patient's body. Such a neurostimulator device system may comprise a stimulus generator, attached to an electrical lead having a nerve electrode coupled to the vagus nerve.

However, depending upon a patient population or a particular disease, efficacy of the VNS therapy may vary significantly. For instance, VNS efficacy for treatment resistant epilepsy and depression may be generalized as a first percentage of patient population having significant improvement. A second percentage of patient population may be characterized as having some improvement. The remaining percentage of patient population may experience little improvement. There is a need to improve the efficacy of VNS therapy for certain treatments. Further concerns include reducing any side effects during stimulation.

Neurostimulation has demonstrated the potential to treat a wide variety of neurological disorders; however, there remains a need to increase the breadth of disorders treatable by neurostimulation.

### SUMMARY OF THE INVENTION

In one aspect, the present invention comprises a method for providing a neurostimulation therapy to a patient. The method includes generating an electrical biasing signal comprising a pulsed electrical signal defined by a plurality of parameters including at least one parameter selected from the group consisting of a voltage magnitude, a current magnitude, a pulse width, a pulse period, an on-time and an off-time. At least one of the parameters comprises a random value that varies within a defined range. The method also comprises applying the electrical biasing signal to a neural structure of the patient.

In some embodiments, the defined random range for the voltage magnitude may comprise a programmed range within the range of −15.0 to 15.0 volts, the defined random range for the current may comprise a programmed range within the range of from −8.0 to 8.0 milliamps, the defined random range for the pulse width may comprise a programmed range within the range of from 1 microsecond to 1 second, the defined random range for the pulse period may comprise a programmed range within the range of from 1 microsecond to 1 second, the defined random range for the on-time may comprise a programmed range within the range of from 1 second to 24 hours, and the defined random range for the off-time may comprise a programmed range within the range of from 1 second to 24 hours.

In some methods of the invention, the neural structure may comprise an intrinsic neural signal, and the pulsed electrical signal may operate either to attenuate or to amplify the intrinsic neural signal. The method may comprise changing a threshold of interpretation for the intrinsic neural signal to enable the patient's brain to interpret the intrinsic neural signal in a desired manner, where changing a threshold comprises raising a threshold of interpretation or lowering a threshold of interpretation. The method may further comprise modulating the intrinsic neural signal with the pulsed electrical signal to block transmission of the intrinsic neural signal along the neural structure.

In some embodiments, methods of the invention may comprise detecting an intrinsic neural signal on the neural structure. In some methods, the detected intrinsic neural signal may be compared to a threshold of intrinsic neural activity, and the electrical biasing signal that is generated may depend upon the outcome of the comparing step. The electrical biasing signal may bias the intrinsic neural signal from either a sub-threshold or a supra-threshold level sufficiently to allow the intrinsic neural signal to cross a threshold of interpretation for the brain.

In some embodiments, the defined random range for at least one parameter of the pulsed electrical signal may comprise an upper limit and a lower limit, and at least one of the upper limit and lower limit may be defined based upon a pain threshold of the patient.

In another embodiment, the method may further comprise generating a plurality of afferent action potentials on the neural structure to enhance interpretation of the intrinsic neural signal by the brain of the patient.

In another embodiment, methods of the invention may comprise providing a pulsed electrical signal comprising a current magnitude that is random and varies within a range within the range of from −8.0 milliamps to 8.0 milliamps. In another embodiment, the current magnitude may be random and vary within a range within the range of from −3.0 to 3.0 milliamps. In another embodiment, the pulsed electrical signal may comprise a random pulse width that varies within a range within the range of from 1 microsecond to 1 second. In still another embodiment, methods of the invention may comprise providing a pulsed electrical signal comprising a current magnitude that is random and varies within a first defined range and a pulse width that is random and varies within a second defined range.

In some methods of the invention, the pulsed electrical signal comprises a pulse period that is random and varies within a range within the range of from 1 microsecond to 1 second. In some embodiments of the invention the pulsed electrical signal comprises a voltage magnitude that is random and varies within a range within the range of −15.0 volts to 15.0 volts.

In some embodiments, the pulsed electrical signal comprises an on-time and an off-time, and at least one of the on-time and off-time may comprise a random value that varies within a defined range. In particular embodiments, at least one of the on-time or the off-time may vary within a range within the range of from 1 second to 24 hours.

In some methods, the invention may comprise a first time interval in which at least one of the voltage magnitude, current magnitude, pulse width, pulse period, on-time and off-time comprises a random value that varies within a defined range, and a second time interval in which the at least one parameter that is random in the first time interval is non-random.

In some embodiments, the random value varies within a defined range on a pulse-to-pulse basis. In other embodiments, the random value varies within a defined range on a burst-to-burst basis.

In a further embodiment, the neural structure to which the electrical biasing signal is applied comprises a cranial nerve of the patient. The cranial nerve may comprise a vagus nerve. In other embodiments of the invention, the neural structure comprises a structure within the patient's brain. In still further embodiments, the neural structure comprises a spinal cord structure of the patient. The neural structure may in some embodiments comprise a sympathetic nerve.

In certain embodiments of the invention, the electrical biasing signal comprises a pulsed noise signal.

In another aspect, methods of the invention may further comprise providing at least one electrode, coupling the at least one electrode to the neural structure, providing an electrical signal generator, coupling the electrical signal generator to the at least one electrode, generating the electrical biasing signal using the electrical signal generator, and applying the electrical biasing signal to the at least one electrode.

In another aspect, the invention may comprise a method of providing a neurostimulation therapy to a patient that comprises generating an electrical biasing signal comprising a pulsed electrical signal defined by a plurality of parameters comprising at least a current magnitude, a pulse width, and a pulse period, in which the pulse period comprises a random value that varies within a defined range, and applying the electrical biasing signal to a neural structure of the patient.

In some embodiments, the method may comprise a first time interval in which the pulse period comprises a random value that varies within a defined range, and a second time interval wherein the pulse period comprises a non-random value. In some embodiments, the pulse period comprises a random value that varies within a defined range on a pulse-to-pulse basis. In other embodiments, the pulse period comprises a random value that varies within the defined range on a burst-to-burst basis.

In some embodiments of the invention, the current magnitude comprises a constant magnitude. In other embodiments, the current magnitude comprises a random value that varies within a defined range. In some embodiments, the pulse width comprises a random value that varies within a defined range.

In some embodiments, the electrical biasing signal comprises a continuous electrical signal.

Some embodiments of the methods of the invention, the pulsed electrical signal further comprises an on-time and an off-time, and the on-time and the off-time may comprise a random value or a constant value.

In some embodiments, the neural structure comprises an intrinsic neural signal, and the methods of the invention further comprise detecting the intrinsic neural signal on the neural structure.

In some embodiments, methods of the invention further comprise comparing the detected intrinsic neural signal to a threshold of intrinsic neural activity. The pulsed electrical signal further comprises an on-time and an off-time, which each comprise one of a random value that varies within a defined range and a constant value. At least one of the on-time and off-time may depend upon the outcome of the comparing step.

In another aspect, the invention comprises a method of providing a neurostimulation therapy to a patient. The method comprises generating an electrical biasing signal comprising a pulsed electrical signal defined by a plurality of parameters comprising a constant current magnitude, a constant pulse width, an on-time and an off-time, and at least one of the on-time and the off-time comprises a random value that varies within a defined range. The method also comprises applying the electrical biasing signal to a neural structure of a patient.

In some embodiments, the method further comprises a first time interval in which at least one of the on-time and the off-time comprises a random value that varies within a defined range, and a second time interval in which at least one of the on-time and the off-time comprises a non-random value. In other embodiments, the on-time comprises a random value that varies within a first defined range, and the off-time comprises a random value that varies within a second defined range.

In some embodiments, the plurality of parameters defining the pulsed electrical signal further comprises a frequency selected from the group consisting of a controlled frequency, a random frequency within a defined frequency range, and a swept frequency within a defined range. In other embodiments, the plurality of parameters further comprises a pulse period selected from the group consisting of a controlled pulse period and a random pulse period that varies within a defined range.

In another aspect, the invention comprises a method of providing a neurostimulation therapy to a patient. The method comprises generating an electrical biasing signal comprising an electrical signal defined by a plurality of parameters comprising a current magnitude and at least one of an on-time and an off-time. At least one of the current magnitude, the on-time and the off-time comprises a random value that varies within a defined range. The method further comprises applying the electrical biasing signal to a neural structure of the patient.

In another embodiment, the method further comprises a first time interval in which at least one of the current magnitude, the on-time and the off-time comprises a random value that varies within a defined range, and a second time interval in which the at least one parameter comprising a random value in the first time interval comprises a non-random value.

In some embodiments, the electrical signal in methods of the invention comprises a non-pulsed electrical signal. The electrical signal may in some embodiments comprise a charge-balanced electrical signal. In some embodiments, the electrical biasing signal comprises a noise signal having a random current magnitude that varies within a range within the range of from −8.0 to 8.0 milliamps.

In some embodiments, the on-time is random and varies within a range within the range of from 1 second to 24 hours, and said the off-time is also random and likewise varies within a range within the range of from 1 second to 24 hours.

In another aspect, the invention comprises a method of providing a neurostimulation therapy to a patient. The method comprises generating an electrical biasing signal comprising a non-pulsed, continuous electrical signal defined by at least a current magnitude, in which the current magnitude is random and varies within a range within the range of from −8.0 to 8.0 milliamps. The method also comprises applying the electrical biasing signal to a neural structure of the patient.

In another aspect, the invention comprises a method of providing a neurostimulation therapy to a patient comprising generating an electrical biasing signal comprising an electrical noise signal, and applying the electrical biasing signal to a neural structure of the patient that is selected from the group consisting of a cranial nerve, a brain structure, a spinal cord structure, and a sympathetic nerve structure.

In another embodiment, the electrical noise signal comprises a noise signal selected from the group consisting of a zero-mean, pseudo-random, or Gaussian noise signal.

In one aspect, the present invention comprises a method for providing an electrical neurostimulation therapy to a patient. The method includes applying an electrical biasing signal to a cranial nerve to bias an intrinsic neural signal on the cranial nerve. The electrical biasing signal may be sufficient to cause the intrinsic neural signal to reach a threshold stimulus for the brain of the patient.

In a further aspect, a method of treating a patient with neurostimulation comprises detecting an intrinsic neural signal on a cranial nerve of the patient. The method further comprises generating an electrical biasing signal in response to the detected intrinsic neural signal and applying the electrical biasing signal to the cranial nerve to bias the intrinsic neural signal on the cranial nerve, thereby providing electrical neurostimulation therapy to the patient.

In another aspect of the present invention, a method of providing electrical neurostimulation therapy to a patient comprises applying a bias stimulus to an electrode coupled to a selected cranial nerve of the patient. The method further comprises enabling the brain to interpret an intrinsic neural signal in response to the bias stimulus.

In another aspect of the present invention, a method of treating a patient by an implanted neurostimulator device comprises coupling the implanted neurostimulator device to a vagus nerve of the patient. The method further comprises applying a bias stimulus to the vagus nerve and enabling the brain to interpret an intrinsic neural signal of the vagus nerve in response to the bias stimulus.

In another aspect, the invention comprises a neurostimulation system for treating a patient with a medical condition. The system comprises a stimulus generator to generate an electrical biasing signal for at least a target portion of a neural structure of a patient. The electrical biasing signal comprises a pulsed electrical signal defined by at least one parameter selected from the group consisting of a voltage magnitude, a current magnitude, a pulse width, a pulse period, an on-time and an off-time. At least one of the voltage magnitude, current magnitude, pulse width, pulse period, on-time and off-time comprises a random value that varies within a defined range. The system also comprises at least one electrode coupled to said stimulus generator and to a neural structure of the patient, and a controller operatively coupled to the stimulus generator. The controller is adapted to apply the electrical biasing signal to the neural structure to bias an intrinsic neural signal on the neural structure.

In one embodiment, the system further comprises a random data generator for generating said random value for said at least one parameter. The system may also comprise a memory for storing the defined range for the random value.

In another embodiment, the neural structure to which the electrode is coupled comprises a cranial nerve, a sympathetic nerve, a spinal cord structure, and a structure within the patient's brain.

In a further embodiment, the at least one parameter of the electrical biasing signal comprises a voltage magnitude that is random and varies within a range within the range of from −15.0 volts to 15.0 volts. In another embodiment, the at least one parameter of the electrical biasing signal comprises a current magnitude that is random and varies within a range within the range of from −8.0 milliamps to 8.0 milliamps. The current magnitude may comprise a random value that varies within a range within the range of from −3.0 milliamps to 3.0 milliamps.

In one embodiment, the at least one parameter of the electrical biasing signal comprises a pulse width that is random and varies within a range within the range of from 1 microsecond to 1 second. In another embodiment, the at least one parameter of the electrical biasing signal comprises a pulse period that is random and varies within a range within the range of from 1 microsecond to 1 second.

In a further embodiment, the at least one parameter of the electrical biasing signal comprises a current magnitude that is random and varies within a first defined range, and a pulse width that is random and varies within a second defined range.

In one embodiment, the at least one parameter of the electrical biasing signal comprises an on-time that is random and varies within a range within the range of from 1 second to 24 hours. In another embodiment, the at least one parameter of the electrical biasing signal comprises an off-time that is random and varies within a range within the range of from 1 second to 24 hours

Neurostimulation systems of the present invention may, in other embodiments, further comprise a sensor for detecting an intrinsic neural signal on said neural structure. The system may further comprise a signal analysis unit for comparing the detected intrinsic neural signal to a threshold of intrinsic neural activity. The controller may further comprise a switching network for applying the electrical biasing signal to the neural structure in response to the signal analysis unit. In a further embodiment, the controller may comprise a stimulation selection unit for adjusting at least one of the parameters in response to the comparing step.

The defined range for the at least one parameter may, in some embodiments of the system, comprise an upper limit and a lower limit. At least one of the upper limit and the lower limited may be defined based upon a pain threshold of the patient.

In some embodiments, the electrical biasing signal of the neurostimulation system may comprise a pulsed noise signal.

In a particular embodiment, the at least one electrode comprises a pair of electrodes for contacting the neural structure for direct stimulation. In another embodiment, the neurostimulation system may further comprise a communication interface and a programming unit in communication with the communication interface. The programming unit is capable of programming the at least one parameter defining the electrical biasing signal.

In one embodiment of the neurostimulation system, the pulsed electrical signal further comprises a first time interval in which at least one of the voltage magnitude, current magnitude, pulse width, pulse period, on-time and off-time comprises a random value that varies within a defined range, and a second time interval in which the at least one parameter that is random in the first time interval is non-random.

In some embodiments of the neurostimulation system, the random value varies within a defined range on a pulse-to-pulse basis. In other embodiments, the random value varies within a defined range on a burst-to-burst basis.

In another aspect, the invention comprises a neurostimulator for providing an electrical stimulation therapy to a patient. The neurostimulator comprises a stimulus generator to generate an electrical biasing signal for an intrinsic neural signal in a neural structure of the patient. The electrical biasing signal comprises a pulsed electrical signal defined by a plurality of parameters comprising at least a current magnitude, a pulse width, and a pulse period. The pulse period comprises a random value that varies within a defined range. The neurostimulator also comprises at least one electrode coupled to the stimulus generator and the neural structure, and a controller coupled to the stimulus generator and adapted to apply the electrical biasing signal to the neural structure of the patient.

In some embodiments of the neurostimulator, the neural structure may comprise a cranial nerve, a sympathetic nerve, a spinal cord structure, or a structure within the patient's brain.

In one embodiment, the pulsed electrical signal comprises a current magnitude that is a constant magnitude. In other embodiments, the current magnitude comprises a random value that varies within a defined range. In some embodiments, the pulse width comprises a random value that varies within a defined range.

The electrical biasing signal in some neurostimulator embodiments comprises a continuous electrical signal.

In one neurostimulator embodiment, the plurality of parameters defining the pulsed electrical signal further comprises an on-time and an off-time, each of which may comprise a random value or a non-random value.

In one embodiment, the neurostimulator may further comprise a sensor for detecting said intrinsic neural signal on said neural structure. The neurostimulator may also comprise a signal analysis unit for comparing the detected intrinsic neural signal to a threshold of intrinsic neural activity. The controller may comprise a switching network for applying the electrical biasing signal to the neural structure in response to the signal analysis unit. The plurality of parameters defining the pulsed electrical signal may comprise an on-time and an off-time, which may be random or non-random, and the controller may further comprise a stimulation selection unit for adjusting one of the on-time or off-time in response to the signal analysis unit.

In another aspect, the invention comprises a neurostimulator for providing an electrical stimulation therapy to a patient. The neurostimulator comprises a stimulus generator to generate an electrical biasing signal for an intrinsic neural signal in a neural structure of the patient. The electrical biasing signal comprises a pulsed electrical signal defined by a plurality of parameters comprising a constant current magnitude, a constant pulse width, an on-time and an off-time. At least one of the on-time and off-time comprises a random value that varies within a defined range. The neurostimulator also comprises at least one electrode coupled to the stimulus generator and the neural structure, and a controller coupled to the stimulus generator and adapted to apply the electrical biasing signal to the neural structure of the patient.

In one embodiment, the on-time comprises a random value that varies within a first defined range, and the off-time comprises a random value that varies within a second defined range.

In another embodiment, the plurality of parameters defining the pulsed electrical signal further comprises a frequency, which may be a non-random frequency, a random frequency within a defined frequency range, or a swept frequency within a defined range.

In a further embodiment of the neurostimulator, the plurality of parameters defining the pulsed electrical signal further comprises a pulse period. The pulse period may be a constant pulse period or a random pulse period that varies within a defined range.

In another aspect, the invention comprises a neurostimulator for providing an electrical stimulation therapy to a patient. The neurostimulator comprises a stimulus generator to generate an electrical biasing signal for an intrinsic neural signal in a neural structure of the patient. The electrical biasing signal comprises an electrical signal defined by a plurality of parameters comprising a current magnitude and at least one of an on-time and an off-time. At least one of the current magnitude, on-time and off-time comprises a random value that varies within a defined range. The neurostimulator further comprises at least one electrode coupled to said stimulus generator and to said neural structure, and a controller coupled to the stimulus generator and adapted to apply the electrical biasing signal to the neural structure of the patient.

In one embodiment, the electrical signal comprises a non-pulsed electrical signal. In another embodiment, the electrical signal comprises a charge-balanced electrical signal. In a still further embodiment, the electrical biasing signal comprises a noise signal having a random current magnitude that varies within a range within the range of from −8.0 to 8.0 milliamps. In another embodiment, the on-time is random and varies within a range within the range of from 1 second to 24 hours, and the off-time is random and likewise varies within a range within the range of from 1 second to 24 hours.

In another aspect, the invention comprises a neurostimulator for providing an electrical stimulation therapy to a patient. The neurostimulator comprises a stimulus generator to generate an electrical biasing signal for an intrinsic neural signal in a neural structure of the patient. The electrical biasing signal comprises a non-pulsed, continuous electrical signal defined by at least a current magnitude that is random and varies within a range within the range of from −8.0 to 8.0 milliamps. The neurostimulator further comprises at least one electrode coupled to the stimulus generator and to the neural structure, and a controller coupled to the stimulus generator and adapted to apply the electrical biasing signal to the neural structure of the patient.

In another aspect, the invention comprises a neurostimulator for providing an electrical stimulation therapy to a patient. The neurostimulator comprises a stimulus generator to generate an electrical biasing signal comprising an electrical noise signal for biasing an intrinsic neural signal in a neural structure. The neural structure is a structure selected from the group consisting of a cranial nerve, a brain structure, a spinal cord structure, and a sympathetic nerve structure. The neurostimulator further comprises at least one electrode coupled to the stimulus generator and to the neural structure, and a controller coupled to the stimulus generator and adapted to apply the electrical biasing signal to the neural structure of the patient.

In one embodiment, the electrical noise signal comprises a noise signal selected from the group consisting of a zero-mean, pseudo-random, or a Gaussian noise signal.

In still another aspect of the present invention, an implantable medical device, such as a neurostimulator is provided for treating a neurological disease, disorder or condition. The neurostimulator comprises an electrical stimulus generator to generate an electrical stimulation signal for delivery to a cranial nerve. The neurostimulator further comprises a controller operatively coupled to the stimulus generator. The controller may be adapted to apply the electrical stimulation signal to the cranial nerve so as to bias the intrinsic neural signal on the nerve and provide electrical neurostimulation therapy to the patient.

In another aspect, a neurostimulation system is provided for treating a patient with a medical condition. The system comprises an electrical stimulus generator to generate an electrical stimulation signal for at least a target portion of a cranial nerve of the patient. The neurostimulation system may further comprise a controller operatively coupled to the stimulus generator. The controller may be adapted to apply the electrical stimulation signal to the target portion of the cranial nerve to bias an intrinsic neural signal on the cranial nerve.

In yet another aspect, the present invention comprises a computer readable program storage device encoded with instructions for providing an electrical neurostimulation therapy to a patient from an implantable medical device. The instructions in the computer readable program storage device, when executed by a computer, apply an electrical biasing signal to a cranial nerve to bias an intrinsic neural signal on the cranial nerve. The electrical biasing signal may be sufficient to cause the intrinsic neural signal to reach a threshold stimulus for the brain of the patient.

In yet another aspect, the present invention comprises a computer readable program storage device encoded with instructions of providing a neurostimulation therapy to a patient from an implantable medical device. The instructions in the computer readable program storage device, when executed by a computer, generate an electrical biasing signal comprising a pulsed electrical signal defined by a plurality of parameters comprising a current magnitude and a pulse width, wherein at least one of the current magnitude and the pulse width varies randomly from pulse to pulse within a defined range, and apply the electrical biasing signal to a neural structure of a patient.

### BRIEF DESCRIPTION OF THE DRAWINGS

The invention may be understood by reference to the following description taken in conjunction with the accompanying drawings, in which like reference numerals identify like elements, and in which:

FIG. 1 is a stylized schematic representation of an implantable medical device that delivers an electrical stimulus to one or more nerve fibers in a nerve bundle of a nerve trunk for treating a patient with neurostimulation according to one illustrative embodiment of the present invention;

FIG. 2 is a stylized diagram of an implantable medical device implanted into a patient's body for providing electrical stimulation to a vagus nerve, with an external programming user interface, in accordance with an illustrative embodiment of the present invention;

FIG. 3 is a stylized schematic representation of a signal with an applied stochastic bias indicative of that which a neurostimulator of the present invention may apply to the vagus nerve to enable the brain of the patient to interpret the afferent intrinsic neural signal, consistent with one exemplary embodiment of the present invention;

FIGS. 4A-4E are diagrams of various randomized electrical biasing output current signals provided by the implantable medical device of FIGS. 1 and 2, in accordance with various illustrative embodiments of the present invention;

FIG. 5 is a stylized schematic representation of the neurostimulator of FIG. 2, for applying an electrical biasing signal to the vagus nerve, in accordance with one illustrative embodiment of the present invention;

FIG. 6 is a stylized schematic representation of the stimulation controller of FIG. 4, according to one illustrative embodiment of the present invention;

FIG. 7 is a flow chart representation of a method for treating a patient with neurostimulation from an implantable medical device, in accordance with one illustrative embodiment of the present invention;

FIG. 8 is a flow chart representation of a method of applying a bias stimulus to a vagus nerve to enable the brain of the patient to interpret the intrinsic neural signal on the nerve, in accordance with one illustrative embodiment of the present invention; and

FIG. 9 is a flow chart representation of a method of causing intrinsic vagal activity in the intrinsic neural signal from the vagus nerve to clarify and/or correct the nerve stimulation at the brain of the patient for a desired level of interpretation based on the neurostimulation, in accordance with one illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

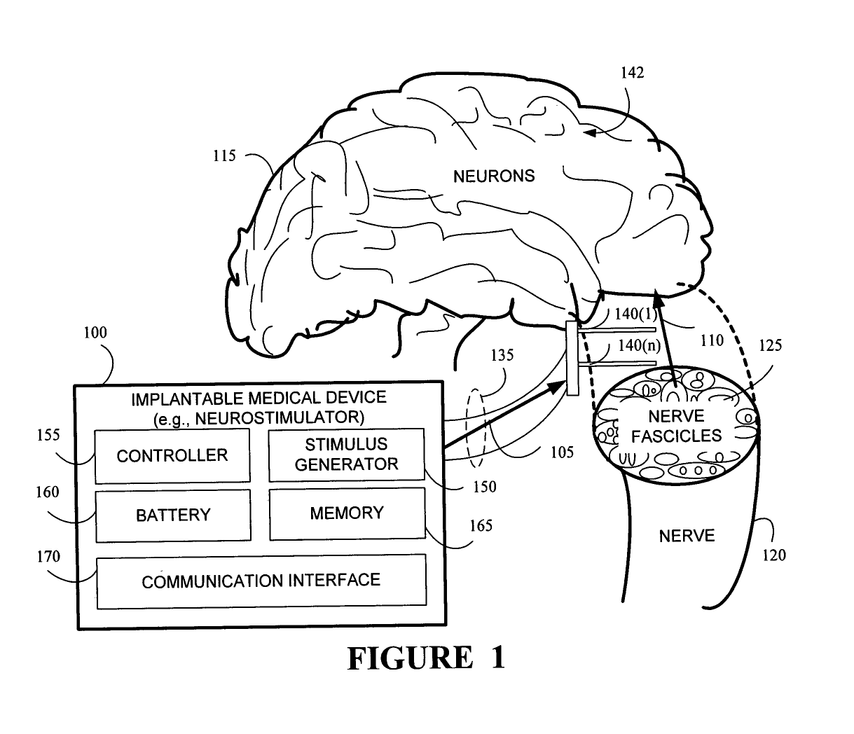

In one embodiment of the present invention, methods, apparatus, and systems provide a bias stimulus to the intrinsic neural activity in a nerve, which is preferably a cranial nerve, and more preferably a vagus nerve. “Intrinsic neural activity” or “intrinsic neural signal” on the nerve refers to the electrical activity (i.e., afferent and efferent action potentials) what are generated solely by the patient's body and environment, and not by applied electrical signals from, e.g., an implanted neurostimulator. Turning now to FIG. 1, a medical device, which is preferably an implantable medical device 100, is illustrated for providing a neurostimulation therapy to a patient, according to one embodiment of the present invention. The implantable medical device 100 may deliver an electrical stimulus 105 to an intrinsic neural signal 110 that travels to the brain 115 of a patient. The nerve 120 or a nerve fascicle 125 within the nerve 120 provides the intrinsic neural signal 110, and the electrical stimulus 105 to the brain 115.

The implantable medical device 100 may modulate the intrinsic neural signal 110 by delivering the electrical stimulus 105 to the nerve 120 via a lead 135 coupled to one or more electrodes 140 (1-n). For example, the electrical stimulus 105 may enhance the intrinsic neural signal 110 by clarifying and/or correcting interpretation by the brain 115 and/or CNS of the intrinsic neural signal 110 from the selected nerve 120.

Consistent with one embodiment, the implantable medical device 100 may be a neurostimulator device capable of treating a disease, disorder or condition by providing electrical neurostimulation therapy to a patient. To this end, the implantable medical device 100 may be implanted in the patient at a suitable location. The implanted medical device 100 may apply the electrical stimulus 105, which may comprise an electrical biasing signal, to the nerve 120 to modulate the intrinsic neural signal 110 of one or more nerve fibers or nerve fascicles 125 within the nerve 120. By applying the stimulus 105 (e.g., an electrical bias stimulus utilizing stochastic resonance), the implantable medical device 100 may treat or control medical, psychiatric or neurological disorders in a patient.

Stochastic resonance (SR) is a mechanism whereby the response of a nonlinear system to a weak input signal is optimized by the presence of a nonzero level of noise. In such a mechanism, the noise plays a constructive role in information transfer. The nonlinear system in a patient may be understood to be the brain 115 and/or CNS receiving information via intrinsic neural signals 110 from one or more nerves 120 or other neural structures (e.g., brain structures, spinal cord structures) and responsively controlling or altering physiologic functions in a patient. In one embodiment, electrical stimulus 105 having one or more random characteristics, including but not limited to voltage magnitude, current magnitude (i.e., amplitude), pulse width, pulse period and pulse polarity, may be used to “amplify” the effect of small intrinsic neural signals 110. In other words, stochastic resonance is a form of electrical biasing stimulus that when applied to the nerve 120, nerve fascicle 125 or other neural structure, may provide a means to enhance the interpretation by the brain 115 and/or CNS of the information contained in the intrinsic neural signals 110 in patients suffering from insufficient or excessive intrinsic neural signals 110. Prior art has demonstrated that the application of an appropriate level of noise to mechanoreceptor cells may enhance the detection of mechanical forces by those mechanoreceptor cells. In this prior art, the nonlinear system is the mechanical force detection threshold of the mechanoreceptor cell. In contrast, this invention applies the bias electrical stimulus 105 to one or more neural structures, such as nerves 120 or nerve fascicles 125 to enhance the interpretation of information contained in the intrinsic neural signals 110, wherein the nonlinear system comprises the brain 115 and/or CNS and its associated inputs and outputs.

Because the brain 115 controls, mediates, or alters physiologic functions in a patient in response to the interpretation of intrinsic neural signals 110, faulty interpretation may lead to faulty control, mediation, or alteration of physiologic functions. This may result in one or more medical, psychiatric, or neurological disorders in a patient, or the insufficient mediation of one or more existing disorders. It is an objective of the present invention to reduce the potential for faulty interpretation of the intrinsic neural signals 110.

Using the bias electrical stimulus 105, in one embodiment, the implantable medical device 100 may improve the treatment of neurological, neuropsychiatric, or neurologically related diseases or disorders by improving the quality of an intrinsic neural signal 110 as perceived by the brain 115. For example, providing electrical bias stimulation for at least one of the trigeminal, glossopharyngeal, and vagus nerves, or other parasympathetic and/or sympathetic nerves, may improve the ability of the brain 115 to interpret intrinsic neural signals 110 in patients suffering from one or more neurological, neuropsychiatric, or neurologically mediated diseases or disorders. Without being bound by theory, in contrast to conventional vagus nerve stimulation (VNS) which introduces extrinsic signals into the brain 115 which may target regions or activities in the brain 115 that directly affect improvements in neuropsychiatric disorders, the implantable medical device 100 of the present invention is intended to modulate intrinsic neural signals 110 to affect their perceptibility by the brain 115. Rather than simply inducing neural activity or central nervous system (CNS) responses in the brain 115 using conventional VNS, which may be considered a method which introduces new neural “information”, the implantable medical device 100 may improve deficient, excessive, or ambiguous intrinsic neural activity 110 through the use of “informationless” bias electrical stimulus 105 having random characteristics. In methods and systems of the present invention, instead of providing new information content via the electrical stimulus 105, the information is intended simply to clarify the existing information content already present in the nerve, enabling the brain to perceive the information content of signals otherwise not perceptible.

Many neurologically mediated disorders may result from faulty interpretation or perception of afferent intrinsic neural signals (e.g., vagal visceral sensory information). By applying the electrical stimulus 105 to the vagus nerve, the implantable medical device 100 may significantly enhance sensory sensitivity in the brain 115. Additionally or alternatively, the stimulus 105 may significantly enhance the interpretation of the sensory or electrical, existing or intrinsic neural or vagal activity by the brain 115. This enhanced sensory sensitivity and/or interpretation of the activity may substantially improve efficacy of neurostimulation therapy. Essentially all disorders that may be impacted by neural signals, such as the intrinsic neural signal 110, may benefit from the use of the stimulus 105.

In the case of bulimia nervosa, for example, vagal activity may play a significant role in regulating binge/purge desires of the patient. Excessive or insufficient vagal activity (or reduced brain sensitivity to vagal activity) may contribute to those desires. Similarly, for depression, vagal activity and/or sensitivity may play a significant role in regulating mood, as implied by the correlation of depression and reduced heart rate variability. A similar correlation between suppressed or excessive vagal activity with other disorders may also exist. Using embodiments of the present invention, the implantable medical device 100 may substantially increase the efficacy of neurostimulation therapy in treating a wide range of diseases, disorders and conditions. Embodiments of the present invention may significantly reduce a side effect related to the nerve stimulation.

Although the implantable medical device 100 is described preferably as implantable, a person of ordinary skill in the art would recognize that the present invention is not so limited. For example, in one alternative embodiment, the medical device may be partially implantable, such as an implantable electrode with a non-implantable power and control source. In another alternative embodiment, the medical device may be fully non-implantable, such as a transcutaneous stimulation device.

Implantable medical devices 100 that may be used in the present invention include any of a variety of electrical stimulation devices, such as a neurostimulator capable of stimulating a neural structure in a patient, especially for stimulating a patient's cranial nerve such as a vagus nerve. Although the implantable medical device 100 is described in terms of cranial nerve stimulation, and particularly vagus nerve stimulation (VNS), a person of ordinary skill in the art would recognize that the present invention is not so limited. For example, the implantable medical device 100 may be applied to the stimulation of other cranial nerves, such as the trigeminal and/or glossopharyngeal nerves, or other neural tissue, such as one or more brain structures of the patient, spinal nerves, and other spinal structures. In one alternative embodiment, the invention may be implemented in a spinal cord stimulator (SCS). In another alternative embodiment, the invention may be implemented in a brain stimulator such as a deep brain stimulation (DBS) system.

In the generally accepted clinical labeling of cranial nerves, the tenth cranial nerve is the vagus nerve, which originates from the stem of the brain 115. The vagus nerve passes through foramina of the skull to parts of the head, neck and trunk. The vagus nerve branches into left and right branches, or vagi, upon exiting the skull. Left and right vagus nerve branches include both sensory and motor nerve fibers. The cell bodies of vagal sensory nerve fibers are attached to neurons located outside the brain 115 in ganglia groups, and the cell bodies of vagal motor nerve fibers are attached to neurons 142 located within the gray matter of the brain 115. The vagus nerve is a parasympathetic nerve, part of the peripheral nervous system (PNS). Somatic nerve fibers of the cranial nerves are involved in conscious activities and connect the CNS to the skin and skeletal muscles. Autonomic nerve fibers of these nerves are involved in unconscious activities and connect the CNS to the visceral organs such as the heart, lungs, stomach, liver, pancreas, spleen, and intestines. Accordingly, to provide vagus nerve stimulation (VNS), a patient's vagus nerve may be stimulated unilaterally or bilaterally in which a stimulating electrical signal is applied to one or both branches of the vagus nerve, respectively.

Implantable medical device 100 may comprise a stimulus generator 150 and a controller 155 operatively coupled thereto for controlling the nerve stimulation. The stimulus generator 150 may generate the electrical stimulus 105, and the controller 155 may be adapted to apply the electrical stimulus 105 to the cranial nerve 120 to bias the intrinsic neural signal 110 and provide electrical neurostimulation therapy to the patient. The controller 155 may direct the stimulus generator 150 to generate an electrical biasing signal to stimulate the vagus nerve.

To generate the electrical stimulus 105, the implantable medical device 100 may further include a battery 160, a memory 165 and a communication interface 170. More specifically, the battery 160 comprises a power-source battery that may be rechargeable. The battery 160 provides power for the operation of the implantable medical device 100, including electronic operations and the stimulation function. The battery 160, in one embodiment, may be a lithium/thionyl chloride cell or, in another embodiment, a lithium/carbon monofluoride cell. The memory 165, in one embodiment, is capable of storing various data, such as operation parameter data, status data, and the like, as well as program code. The communication interface 170 is capable of providing transmission and reception of electronic signals and/or information to and from an external unit. The external unit may be a device that is capable of programming the implantable medical device 100.

The implantable medical device 100 may be a single device or a pair of devices, is implanted and electrically coupled to the lead(s) 135, which are in turn coupled to the electrode(s) 140 implanted on the left and/or right branches of the vagus nerve, for example. In one embodiment, the electrode(s) 140 (1-n) may include a set of stimulating electrode(s) separate from a set of sensing electrode(s). In another embodiment, the same electrode may be deployed to stimulate and to sense. A particular type or a combination of electrodes may be selected as desired for a given application. For example, an electrode suitable for coupling to a vagus nerve may be used. The electrodes 140 preferably comprise a bipolar stimulating electrode pair. Persons of ordinary skill in the pertinent art will appreciate that many electrode designs could be used in the present invention.

Using the electrode(s) 140(1-n), the stimulus generator 150 may apply a predetermined sequence of electrical pulses to the selected cranial nerve 120 to provide therapeutic neurostimulation for the patient with a disease or a disorder. A non-pulsed electrical signal may also be used. While the selected cranial nerve 120 may be the vagus nerve, the electrode(s) 140(1-n) may comprise at least one nerve electrode for implantation on the patient's vagus nerve for direct stimulation thereof.

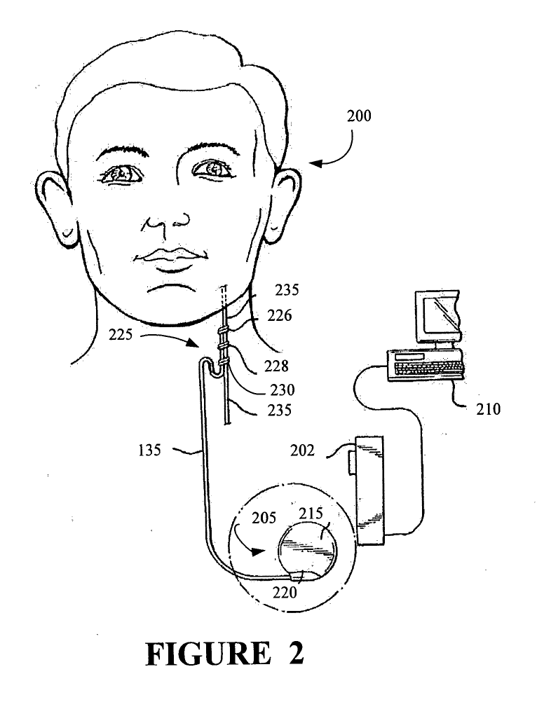

A particular embodiment of the implantable medical device 100 shown in FIG. 1 is illustrated in FIG. 2. As shown therein, an electrode assembly 225, which may comprise a plurality of electrodes such as electrodes 226 and 228, may be coupled to a nerve trunk such as vagus nerve 235 in accordance with an illustrative embodiment of the present invention. Lead 135 is coupled to the electrode assembly 225 and secured, while retaining the ability to flex with movement of the chest and neck, by a suture connection to nearby tissue. The electrode assembly 225 may deliver an electrical signal to the nerve trunk to modulate the intrinsic neural signal 110. Using the electrode(s) 226 and 228, the selected cranial nerve such as vagus nerve 235, may be stimulated within a patient's body 200.

An external programming user interface 202 may be used by a health professional for a particular patient to either initially program and/or to later reprogram the implantable medical device 100, such as a neurostimulator 205. The neurostimulator 205 may include the stimulus generator 150, which may be programmable. To enable physician programming of the electrical and timing parameters of a sequence of electrical impulses, an external programming system 210 may include a processor-based computing device, such as a computer, personal digital assistant (PDA) device, or other suitable computing device.

Using the external programming user interface 202, a user of the external programming system 210 may program the neurostimulator 205. Communication between the neurostimulator 205 and the external programming system 210 may be accomplished using any of a variety of conventional techniques known in the art. The neurostimulator 205 may include a transceiver (such as a coil) that permits signals to be communicated wirelessly between the external programming user interface 202, such as a wand, and the neurostimulator 205.

The neurostimulator 205 having a case 215 with an electrically conducting connector in header 220 may be implanted in the patient's chest in a pocket or cavity formed by the implanting surgeon just below the skin, much as a pacemaker pulse generator would be implanted, for example. A stimulating nerve electrode assembly 225, preferably comprising an electrode pair, is conductively connected to the distal end of an insulated electrically conductive lead 135, which preferably comprises a pair of lead wires and is attached at its proximal end to the connector in header 220. The electrode assembly 225 is surgically coupled to a vagus nerve 235 in the patient's neck. The electrode assembly 225 preferably comprises a bipolar stimulating electrode pair 226 and 228, such as the electrode pair described in U.S. Pat. No. 4,573,481 issued Mar. 4, 1986 to Bullara, which is hereby incorporated by reference herein in its entirety. Persons of skill in the art will appreciate that many electrode designs could be used in the present invention. The two electrodes 226 and 228 are preferably wrapped about the vagus nerve, and the electrode assembly 225 secured to the nerve 235 by a spiral anchoring tether 230 such as that disclosed in U.S. Pat. No. 4,979,511 issued Dec. 25, 1990 to Reese S. Terry, Jr. and assigned to the same assignee as the instant application.

In one embodiment, the open helical design of the electrode assembly 225 (described in detail in the above-cited Bullara patent), which is self-sizing and flexible, minimizes mechanical trauma to the nerve and allows body fluid interchange with the nerve. The electrode assembly 225 conforms to the shape of the nerve, providing a low stimulation threshold by allowing a large stimulation contact area. Structurally, the electrode assembly 225 comprises two electrode ribbons (not shown), of a conductive material such as platinum, iridium, platinum-iridium alloys, and/or oxides of the foregoing. The electrode ribbons are individually bonded to an inside surface of an elastomeric body portion of two spiral electrodes, which may comprise two spiral loops of a three-loop helical assembly.

In one embodiment, the lead assembly 230 may comprise two distinct lead wires or a coaxial cable whose two conductive elements are respectively coupled to one of the conductive electrode ribbons. One suitable method of coupling the lead wires or cable to the electrodes comprises a spacer assembly such as that depicted in U.S. Pat. No. 5,531,778 issued Jul. 2, 1996, to Steven Maschino, et al. and assigned to the same Assignee as the instant application, although other known coupling techniques may be used. The elastomeric body portion of each loop is preferably composed of silicone rubber, and the third loop acts as the anchoring tether for the electrode assembly 225.

In one embodiment, the electrode(s) 140 (1-n) of implantable medical device 100 (FIG. 1) may sense or detect any target parameter in the patient's body 200. For example, an electrode 140 coupled to the patient's vagus nerve 235 may detect the intrinsic neural signal 110. The electrode(s) 140 (1-n) may sense or detect an electrical signal (e.g., a voltage indicative of intrinsic neural electrical activity). Other sensors, such as a pressure transducer, an acoustic element, a photonic element (i.e., light emitting or absorbing), a blood pH sensor, a blood pressure sensor, a blood sugar sensor, a body movement sensor (e.g., an accelerometer), or any other element capable of providing a sensing signal representative of a patient's body parameter may be employed.

In one embodiment, the neurostimulator 205 may be programmed to deliver an electrical biasing signal continuously, periodically at regular time intervals (e.g., every five minutes), or intermittently at irregular time intervals (e.g., on demand or according to circadian rhythms). Neurostimulation has frequently been delivered as a pulsed electrical signal in discrete stimulation periods known as pulse bursts, which constitute a series of controlled pulses having a programmed, non-random and constant current, e.g., 1 milliamp, a programmed frequency, e.g., 30 Hz, a programmed pulse width, e.g., 500 microseconds, a programmed current polarity, e.g., current flow from electrode 226 to electrode 228, for a period of time, e.g., 30 seconds. The period of time in which a stimulation signal is delivered (30 seconds in the example) is referred to herein as on-time. Bursts are typically separated from adjacent bursts by another period of time, e.g. 5 minutes. The period of time between delivery of stimulation signals (5 minutes in the example) is referred to herein as off-time. In prior art embodiments, the current, pulse width, polarity, on-time and off-time are programmed as constant, non-random values. Ramping of the current or voltage over the first few seconds or pulses of a pulse burse is sometimes employed to avoid pain which can be associated with having the initial pulses of a burst at full amplitude. The ramping signal comprises a varying but non-random value, and the remainder of the pulse burst is both constant and non-random. The frequency, which is determined by a plurality of similar adjacent pulse-to-pulse intervals, is also generally a constant value, although it is known to employ a swept or randomly set value. A pulse-to-pulse interval is referred to herein as a pulse period, and is distinct from frequency in that a pulse period is independent of adjacent pulse periods, whereas a frequency, by definition, requires a plurality of similar adjacent pulse periods.

A continuous signal, as used herein, refers to an electrical signal without a distinct on-time and off-time. A continuous signal may be delivered without a distinct on-time and off-time as either a pulsed signal having a constant or random pulse period or frequency, or as a purely continuous signal with no break in current flow at all (although other parameters, such as current magnitude and polarity, may vary within the signal). A non-pulsed signal, as used herein, refers to a signal in which a current is always being delivered during the on-time period, as distinct from a pulsed signal in which flow of current during an on-time period is separated by short periods (typically milliseconds or seconds) of no current flow. It should be noted that non-pulsed signals may be delivered according to a programmed or random on-time and off-time (for example, to allow a recovery/refractory period for the neural tissue stimulated). However, unless the on-time periods have breaks in current flow within each on-time period, the signal remains a non-pulsed signal as used herein.

One or more parameters in the electrical biasing signal may be allowed to randomly vary on either a pulse-to-pulse basis or (for non-continuous signals) on a burst-to-burst basis. In certain embodiments, a parameter may vary randomly from pulse to pulse either within a pulse burst (which may equivalently be referred to as a pulse train) or continuously (where no on-time and off-time is present). For example, the current magnitude for all pulses within a pulse burst having an on-time of 30 seconds may be allowed to vary randomly from a lower limit of 0.5 milliamps to an upper limit of 2.0 milliamps, followed by an off-time period of five minutes, after which the process is repeated. For continuous signals the same or a different random variation may be allowed to continue indefinitely. In other embodiments, a parameter may be allowed to vary randomly from one pulse burst to another, but remain constant within a pulse burst. Such variations only apply for non-continuous signals having a defined on-time and off-time. For example, a current magnitude may be randomly assigned for a pulse burst as 0.75 milliamps, which is maintained as a constant value for all pulses in the burst (excluding any ramping function at the beginning and/or end of the pulse train), lasting for an on-time of 30 seconds. Following an off-time of 2 minutes, a new current magnitude of 1.25 milliamps may be randomly determined for a second pulse burst (or another value between an upper and a lower limit), and all pulses within the burst are provided with the same magnitude. Both pulse-to-pulse and burst-to-burst randomization and considered to be within the scope of the present invention, so long as at least one parameter comprises a random value, either on a pulse-to-pulse basis or on a burst-to-burst basis.

In addition to burst-to-burst randomization, other stimulation regimes may be employed in which the electrical biasing signal comprises at least one random value in a first time period and a non-random value for a second time period. Alternating periods may be provided in which the signal is randomized and non-randomized. For example, in a first time period of thirty seconds (which may comprise the on-time of a first pulse burst or may simply be a defined first portion of a continuous signal) a pulse width may be allowed to vary randomly between 100 microseconds and 1000 microseconds. In a second time interval (which may comprise the on-time of a second discrete pulse burst or a defined second portion of a continuous signal) the pulse width may be maintained as a constant value of 500 microseconds. In this manner, a mixed random and non-random signal may be provided that has therapeutic benefits to the patient and/or reduces side effects. All such embodiments are considered to be within the scope of the invention.

The neurostimulator 205 may be programmed to initiate an electrical biasing signal upon detection of an event or upon another occurrence to deliver a programmed therapy to the patient based on signals received from one or more sensors indicative of corresponding monitored patient parameters. The electrode(s) 140(1-n), as shown in FIG. 1, may be used in some embodiments of the invention to trigger administration of the electrical stimulation therapy to the vagus nerve 235 via electrode assembly 225. Use of such sensed body signals to trigger or initiate stimulation therapy is hereinafter referred to as “active,” “triggered,” or “feedback” modes of administration. Other embodiments of the present invention utilize a periodic or intermittent stimulus signal applied to neural tissue according to a programmed on/off duty cycle without the use of sensors to trigger therapy delivery. This type of delivery may be referred to as a “passive,” or “non-feedback” therapy mode. Both active and passive electrical biasing signals may be combined or delivered by a single neurostimulator according to the present invention. Either or both modes may be appropriate to treat the particular disorder diagnosed in the case of a specific patient under observation.

The stimulus generator 150 may be programmed using programming software of the type copyrighted by the assignee of the instant application with the Register of Copyrights, Library of Congress, or other suitable software based on the description herein, and a programming wand (external programming user interface 202) to facilitate radio frequency (RF) communication between the external programming system 210 and the stimulus generator 150. The wand 202 and software permit noninvasive communication with the stimulus generator 150 after the neurostimulator 205 is implanted. The wand 202 is preferably powered by internal batteries, and provided with a “power on” light to indicate sufficient power for communication. Another indicator light may be provided to show that data transmission is occurring between the wand 202 and the neurostimulator 205.

In one embodiment, an electrical neurostimulation therapy for a neuropsychiatric disorder may be administered by application of an electrical biasing signal to the vagus nerve 235 of the patient 200. The neuropsychiatric disorder may comprise depression, obsessive-compulsive disorders (OCD), attention deficit/hyperactivity disorders (ADHD), schizophrenia, and borderline personality disorders, by way of nonlimiting example. To this end, the neurostimulator 205 may provide vagus nerve stimulation (VNS) therapy in the patient's neck, i.e., the cervical region. The neurostimulator 205 may be activated manually or automatically to deliver the electrical bias signal to the selected cranial nerve via the electrode(s) 226 and 228. The neurostimulator 205 may be programmed to deliver the bias signal continuously, periodically or intermittently when activated, and the signal may be either pulsed or non-pulsed. At least one parameter defining the stimulation preferably comprises a random value within a defined range.

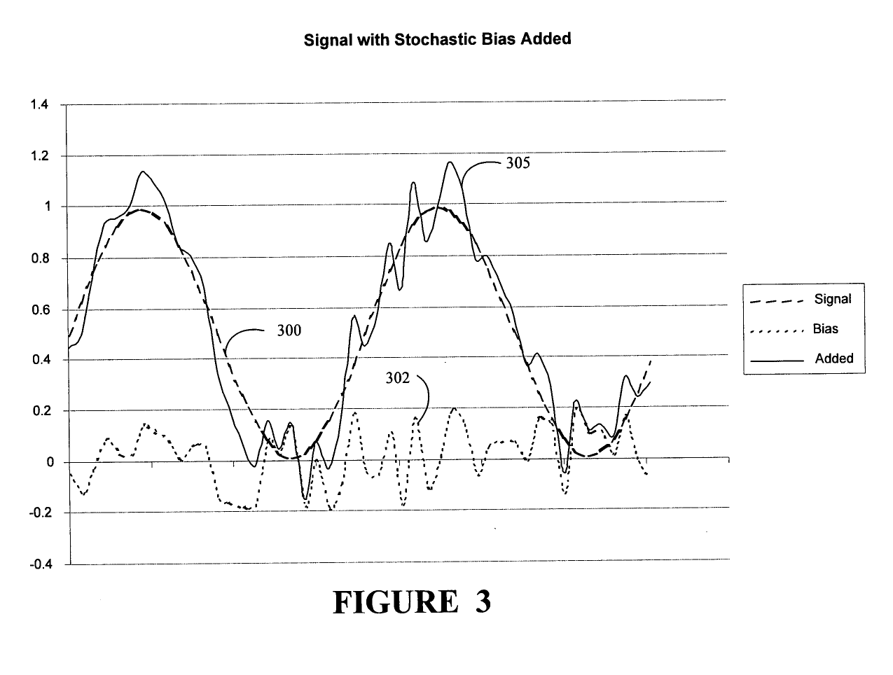

As shown in FIG. 3, the neurostimulator 205 may apply a stochastic electrical biasing signal 302 to an intrinsic neural signal 300 resulting in a modulated signal with stochastic bias added 305 to enhance the intrinsic neural signal in the selected cranial nerve 120, such as the vagus nerve 235, consistent with one exemplary embodiment of the present invention. FIG. 3 is a stylized and generalized representation of the signals and is explanatory of the concepts of this invention. Because nerve 120 may be composed of one or more nerve fibers, the intrinsic neural signal 300 and the modulated signal with stochastic bias added 305 in FIG. 3 may represent either or both of individual neural action potential(s) and the composite information content communicated by the nerve 120 to the brain 115. The vertical scale has been normalized. Use of the stochastic electrical biasing signal 302, resulting in modulated signal 305, may enable the brain 115 to detect and/or interpret otherwise undetectable/interpretable electrical information in the intrinsic neural signal 110. Aging, disease, injury, chemical imbalance, and other disorders may degrade the function of information-carrying nerves 120, the information-interpreting brain 115, and/or the information-producing regions of the body 200 such as, but not restricted to, visceral organs. The degrading of function may include the increase and/or decrease of neural activity and/or detection and/or interpretation thresholds. However, use of the stochastic electrical biasing signal 302 may enhance neural performance of neurons in a cranial nerve, sympathetic nerves, parasympathetic nerves, the spinal cord and/or brain cells that transmit or process the intrinsic neural signal 110.

Instead of using an electrical signal in which the parameters defining the signal are non-random, the electrical stimulus 105 may contain one or more parameters which vary in a random fashion (e.g., white noise) within defined ranges, e.g., a current magnitude range. By adding an electrical biasing signal comprising at least one random parameter to the intrinsic neural signal 110 that is below a threshold of interpretation, the added bias may enable the intrinsic neural signal 110 to cross the threshold of interpretation at which the brain can interpret the intrinsic neural signal. As represented in FIG. 3, if the signal 300 represents information, and a normalized level of 1 represents the threshold of interpretation as activity by the brain, the peaks in signal 300 remain slightly below the threshold. The addition of the bias signal 302 results in the modulated signal 305, in which the peaks cross the threshold. The random variations themselves in modulated signal 305 would be disregarded by the brain as “non-information”; however, the threshold crossings would be interpreted based upon periodic or aperiodic stochastic resonance. The bias signal has effectively “increased” the interpretable information or “decreased” the interpretation threshold of the brain. Referring to the troughs of FIG. 3, if a normalized level of 0 represents the threshold of interpretation as inactivity by the brain, the troughs in signal 300 remain slightly above the threshold. The addition of bias signal 302 results in the troughs of modulated signal 305 crossing below the threshold. The bias signal has effectively “decreased” the interpretable information or “increased” the interpretation threshold of the brain.

Such use of noise to enhance nonlinear system performance is referred to as stochastic resonance since use of signals having one or more random parameters may achieve a larger than expected impact from a small amplitude signal, i.e., the intrinsic neural signal 110. That is, generally the brain 115 over time may adapt to a completely non-random electrical stimulation, and may adapt or begin to disregard it, leading to a loss of efficacy as the brain adapts to the signal. However, the electrical stimulus 105 introduces and/or superimposes random signals over the existing, intrinsic neural signal 110 with relatively small amplitude. In this way, the neurostimulator 205 may enable the brain 115 to detect and/or interpret the intrinsic neural signal 110 from the selected cranial nerve 120, such as the vagus nerve 235.

According to one illustrative embodiment, the stimulus generator 150 may use additive noise (instead of a fixed bias) to generate the electrical stimulus 105 or the electrical biasing signal 302 because the neurons 142 in the brain 115 may adapt to constant or periodic input. The electrical stimulus 105 or the electrical biasing signal 302 may improve the interpretation and availability of composite (multi-axon, multi-purpose) nerve signals, i.e., the intrinsic neural signal 110. The vagus nerve trunk comprises tens of thousands of individual nerve axons, each of which generally conducts an electrical signal in only one direction: either to the brain (afferent fibers) or from the brain (efferent fibers). Thus, the intrinsic neural signal 110 comprises a composite of many individual nerve fibers transmitting information to and from the brain 115. Because of the large amount of neural information it conveys, the vagus nerve 235 may be considered a pipeline or electrical bus for transmission of a diverse collection of information.

Without being bound by theory, instead of improving the performance of axons within the neural tissue itself, the stimulus generator 150 may improve the performance of the brain 115 in interpreting the information present in the intrinsic neural signal 110. Accordingly, the implantable medical device 100 may improve the quality of existing vagal signals as perceived by the brain 115, e.g., the intrinsic neural signal 110 or the interpretation of the signal 110 by the brain 115.

In some patients vagal activity may be insufficient, while in other patients the vagal activity may be hyperactive. Thus, merely providing relatively higher VNS stimulation levels may not necessarily result in improved efficacy for a particular patient. However, by applying stochastic resonance bias, the neurostimulator 205 may bias the intrinsic neural signal 110 to bring it within a band interpretable by the brain 115. Unlike interpretation of a binary threshold of individual sensory cells (producing afferent action potentials for individual fibers) the neurostimulator 205 may bias the intrinsic neural signal 110 to render it sub-threshold or supra-threshold for interpretation. The neurostimulator 205 may eliminate or correct faulty interpretation or erratic availability of neural signals to the brain 115.

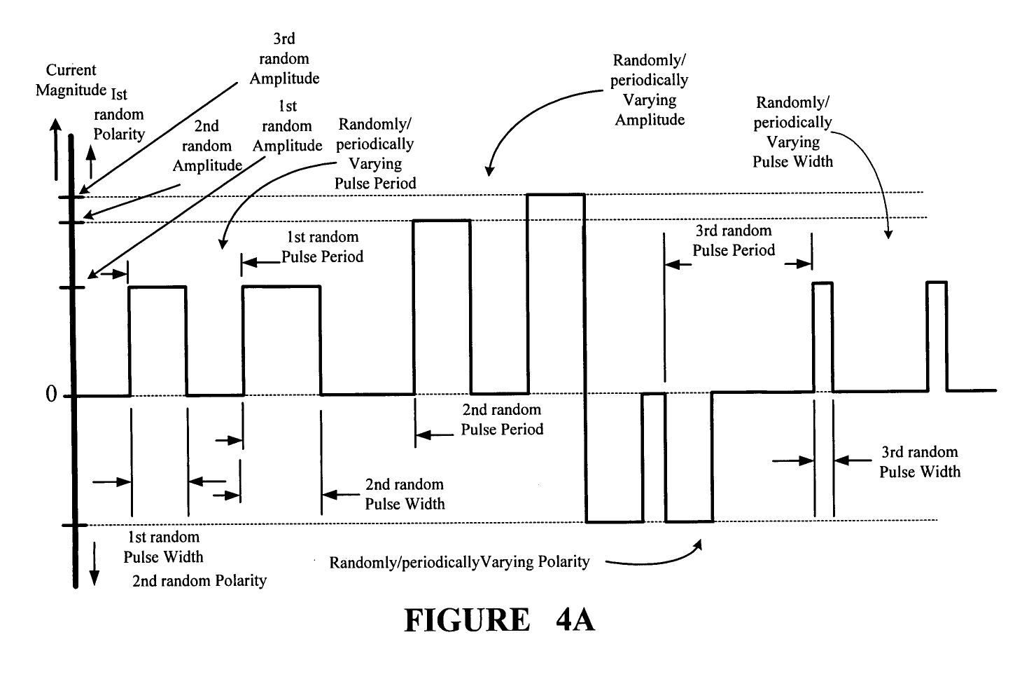

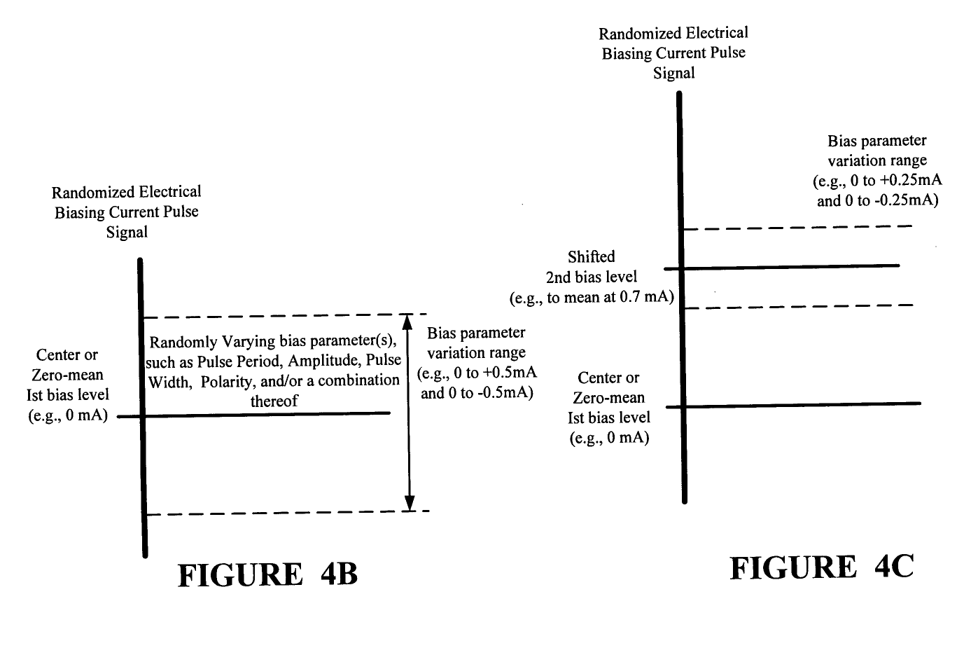

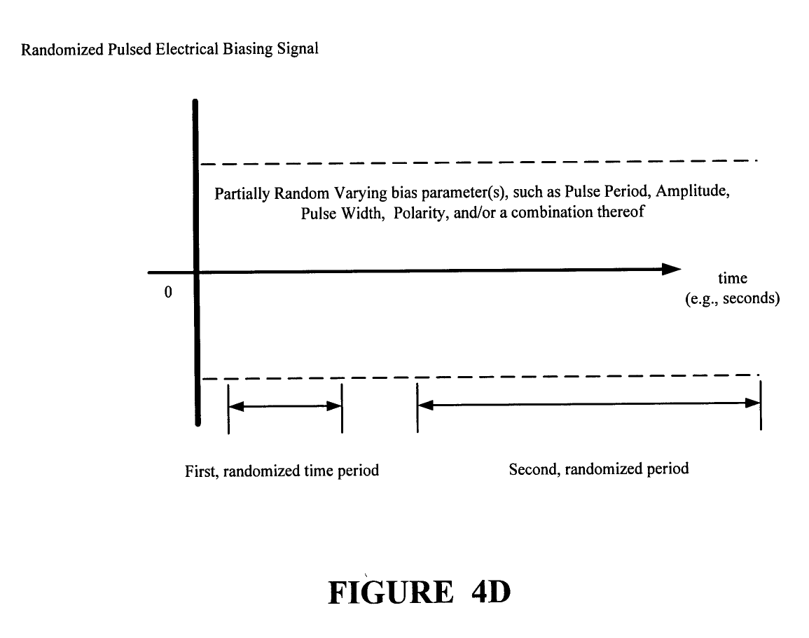

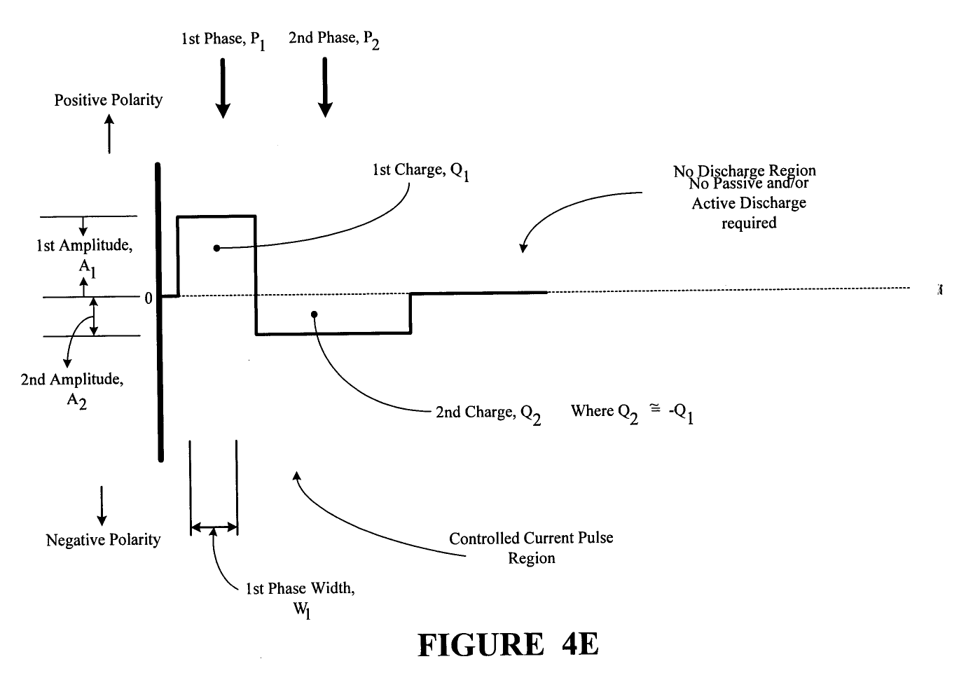

Referring to FIGS. 4A-4E, one embodiment of waveforms illustrates the electrical stimulus 105 or the electrical biasing signal 302 suitable for use in the present invention. The illustrations are presented principally for the sake of clarifying terminology for a plurality of parameters that may be used to define a pulsed electrical signal including a current amplitude, a pulse width, a pulse period (i.e., time interval between the start of adjacent pulses), and a pulse polarity, that may be used by the stimulus generator 150 to generate a pulsed electrical signal. Other parameters (not shown) include signal on-time and signal off-time for non-continuous signals. In embodiments of the present invention, at least one of the voltage amplitude, current amplitude, pulse width, pulse period, pulse polarity, and (for non-continuous signals), signal on-time and signal off-time comprises a random value within a defined range. Examples of the defined range(s) for the operation of the stimulus generator 150 to bias the intrinsic neural signal 110 for clarifying or correcting faulty interpretation or erratic availability of neural signals to the brain 115 is described with reference to FIGS. 4A-4E, which illustrate the general nature, in idealized representation, of pulsed output signal waveforms delivered by the output section of the neurostimulator 250 to electrode assembly 225. One or more biasing parameters may be randomly generated by the stimulus generator 150 to generate a pulsed electrical signal that varies within a defined range.

FIG. 4A illustrates an exemplary pulsed electrical biasing signal provided by embodiments of the present invention. The electrical biasing signal may be a non-continuous signal defined by an on-time and an off-time, or may comprise a continuous signal without discrete pulse bursts (i.e., a signal that does not comprise a distinct on-time and off-time). The electrical biasing signal may alternatively comprise a non-pulsed signal (which may be continuous or non-continuous) with no current breaks during a stimulation period. Whether continuous or non-continuous, the invention comprises signals in which one or more biasing signal parameters are randomly changed for particular pulses in a pulse train (pulse-to-pulse randomization), or alternatively for pulses in adjacent pulse trains (burst-to-burst randomization). Burst-to-burst randomization may comprise changing only the off-time and/or off-time, in which case each of the pulses may be non-random as defined by any of voltage, current, pulse width, pulse period, or frequency, but the duration of adjacent pulse bursts or the interval separating them may comprise a random time interval.

In particular, as FIG. 4A illustrates, the electrical signal pulses in the electrical biasing current signal provided by the neurostimulator 205 may randomly vary in current amplitude, as shown by pulses having first, second a third random amplitudes, respectively, and/or in pulse widths as illustrated by the pulses having first, second and third random pulse widths, respectively. For example, current magnitude of the pulses may be random and vary within any arbitrarily defined range within the range of from −8.0 milliamps (mA) to 8.0 milliamps, such as from −3.0 to 3.0 milliamps or from 0.25 to 1.5 milliamps, with optional charge-balancing. Similarly, pulse widths may be random and vary within any arbitrarily defined range within the range of 1 microsecond to 1 second, such as from 50 to 750 microseconds, or from 200 to 500 microseconds.

In addition to current magnitude and pulse width, FIG. 4A further shows that in some embodiments pulse polarity may vary randomly between a first polarity, indicated by the pulses having a peak above the horizontal zero current line, and a second, opposite polarity, indicated by a peak below the zero current line. FIG. 4A omits, for convenience, any charge-balancing component for a particular pulse. However, it will be understood that each pulse may include a passive or active charge-balancing component. FIG. 4A further illustrates that pulse periods of the electrical pulses also may vary randomly, as illustrated by adjacent pulse pairs having first, second and third random pulse periods. For example, pulse periods of the pulses may be random and vary randomly within any arbitrarily defined range within the range of 1 microsecond to 1 second, for example from 50 microseconds to 200 milliseconds.

While not shown in FIG. 4A, for non-continuous electrical biasing signals defined by an off-time and an off-time, one or both of the on-time and off-time may vary randomly within defined ranges. For example, the on-time defining a pulse burst (or a non-pulsed signal) may be random and vary randomly within any arbitrarily defined range within the range of 1 second to 24 hours and the off-time defining a pulse burst or non-pulsed signal may also be random and vary randomly within any arbitrarily defined range within the range of 1 second to 24 hours.

While FIG. 4A describes parameter randomization for a pulsed electrical biasing signal 302, similar randomization of parameters may be provided for a non-pulsed electrical biasing signal. In particular, while not defined by a pulse width or a pulse interval, a non-pulsed signal may nevertheless be defined by one or more of a current amplitude and a current polarity, and a non-continuous non-pulsed signal may further be defined by an on-time and an off-time. One or more of the foregoing parameters may be randomized for a non-pulsed signal, in similar manner to that described for a pulsed signal, supra.

To generate a randomized electrical biasing current pulse signal, the stimulus generator 150 may randomly and/or periodically vary the bias level and/or the biasing parameter range, as illustrated in FIGS. 4B and 4C. According to one embodiment of the present invention, from one bias level to another bias level, a first biasing parameter range may vary to a second biasing parameter range. The stimulus generator 150 may adjust or shift a first bias level that may be centered on zero-mean in FIG. 4B to a second bias level or mean shown in FIG. 4C. For example, the bias level changes from 0 mA to 0.7 mA and the biasing parameter range from 0 to +0.5 mA and 0 to −0.5 mA to 0 to +0.25 mA and 0 to −0.25 mA. The adjustment of the bias level or the biasing parameter range may depend upon a pain threshold test or a medical condition based feedback.

FIG. 4D and illustrates that signals comprise a randomized signal for a first period of time and non-randomized signals for a second period of time. A biasing parameter may comprise a signal characteristic that is random on a pulse-to-pulse basis and varies within a defined range across a random and/or periodic time interval, but otherwise is non-random. For example, pulse period, amplitude, pulse width, polarity, and/or a combination thereof may randomly vary within a defined range for a first time interval ranging from 1 second to 24 hours. One or more biasing parameters may be randomly varied in first and second periodic ranges during the first time period. For example, the pulse period may be varied randomly for a 30 second period at a value from 50 microseconds to 750 microseconds. In a second time period, the pulse period may comprise a non-random value, for example 500 microseconds for a period of 1 minute. In other embodiments, the ranges of the randomization parameters may comprise a split range. For example, the current magnitude may be allowed to vary on a pulse-to-pulse basis within the ranges of 0.25 to 0.75 milliamps and also in the range of 1.25 to 1.50 milliamps. Accordingly, the current may comprise any value between 0.25 milliamps and 1.50 milliamps except for values comprising 0.76 milliamps to 1.24 milliamps. Such split range randomization may be beneficial for some patients, and is considered to be within the scope of the present invention.

The randomized electrical biasing current signal provided by the neurostimulator 205 may be directed to performing selective activation of various electrodes (described below) to target particular tissue for excitation. An exemplary randomized electrical biasing current pulse signal provided by the neurostimulator 205 is illustrated in FIG. 4A, where randomly varying polarity of a pulse signal is illustrated. In one embodiment, the randomly varying polarity may be employed in conjunction with alternating electrodes for targeting specific tissues. FIG. 4E illustrates an exemplary randomized pulsed electrical biasing signal with a pulse that provides various random phases that correspond to a change in amplitude and a change in polarity. As described above, a phase of a pulse may randomly take on various shapes and current levels, including a current level of zero Amps. In one embodiment, a phase with zero current may be used as a time delay between two current delivery phases of a pulse.

FIG. 4E illustrates a randomized electrical biasing signal and has a first phase that corresponds to a first random amplitude relating to a first charge, Q₁, and a second phase that corresponds to a second random amplitude relating to a second charge, Q₂. In the signal illustrated in FIG. 4E, the second charge Q₂ is substantially equal to the negative value of the first charge Q₁. Therefore, the charges, Q₁ and Q₂, balance each other, reducing the need for active and/or passive discharging of the charges. Hence, the pulse signal illustrated in FIG. 4E is a charge-balanced, randomized electrical biasing current pulse signal. Reducing the need for performing active and/or passive discharge may provide various advantages, such as power savings from the reduction of charge discharge, less circuit requirements, and the like. For example, applying the electrical biasing signal 302 may comprise applying a charge-balanced signal for balancing an electrical charge resulting from the electrical biasing signal 302. For the electrical biasing signal 302, the current magnitude of the pulses may be random and vary within any arbitrarily defined range within the range of −8.0 milliamps to 8.0 milliamps. Various other pulse shapes may be employed in the randomized electrical biasing signal concepts provided by embodiments of the present invention and remain within the scope and spirit of the present invention.

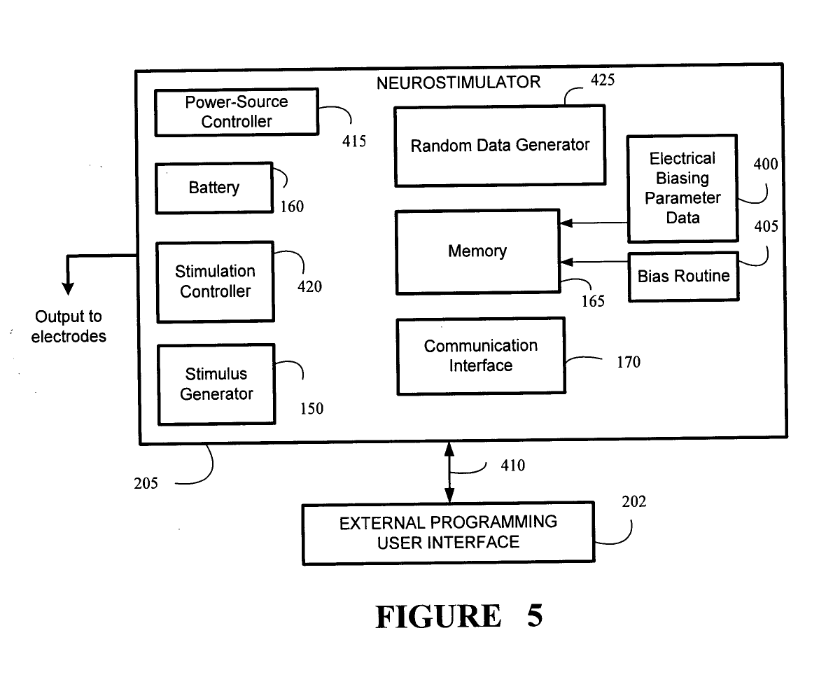

Turning now to FIG. 5, a neurostimulator 205 may be implanted into the patient's body 200 for applying the electrical stimulus 105 or the electrical biasing signal 302 to the vagus nerve 235, in accordance with one illustrative embodiment of the present invention. The neurostimulator 205 comprises the stimulus generator 150, the battery 160, and the memory 165. In one embodiment, the memory 165 may store electrical biasing parameter data 400 and a bias routine 405. The electrical biasing parameter data 400 may include bias parameters with varying amplitudes, durations, polarities, and/or various shapes, and in conjunction with selective electrodes may be employed to hyperpolarize, depolarize, and/or repolarize, various portions of the patient's body to increase neural conduction or neural inhibition.

The bias routine 405 may comprise software and/or firmware instructions to generate the electrical stimulus 105 or the electrical biasing signal 302 that enables electrical neurostimulation for effecting interpretation of the intrinsic electrical neural activity. The bias routine 405 may use the random data generator 425 to provide a randomized electrical biasing signal. For example, based on the electrical biasing parameter data 400, the random data generator 425 may generate random data values or data ranges corresponding to random or pseudo-random numbers provided by the biasing routine 405 for the random biasing parameter data 400. In this way, the stimulus generator 150 may generate the randomized electrical biasing signal. The neurostimulator 205 may then apply the randomized electrical biasing signal to a neural structure, such as the vagus nerve 235 to provide a desired electrical neurostimulation therapy. Utilizing the neurostimulator 205 to bias the intrinsic neural signal 110, as described above, hyper-polarization prior to de-polarization may be performed to allow for an adjustment of nerve stimulation of nerve fibers, and/or other portions of a patient's body.

In accordance with embodiments of the present invention, the neurostimulator 205 may further comprise a communication interface 170. Communications between the external programming user interface 202 and the communication interface 170 may occur via a wireless or other type of communication illustrated generally by a line 410 in FIG. 5. Likewise, the terminals of the battery 160 may be electrically connected to an input side of a power-source controller 415. The power-source controller 415 may comprise circuitry and a processor for controlling and monitoring the power flow to various electronic and stimulation-delivery portions of the neurostimulator 205. The processor in the power-source controller 415 may be capable of executing program code. In one embodiment, the power-source controller 415 is capable of monitoring the power consumption of the neurostimulator 205 and generating appropriate status signals.

The neurostimulator 205 may further comprise a stimulation controller 420 that defines the electrical stimulus 105 to be delivered to the nerve tissue according to parameters that may be preprogrammed into the neurostimulator 205 using the external programming user interface 202. The stimulation controller 420, which may comprise a processor that can execute program code, controls the operation of the stimulus generator 150, which in one embodiment generates the electrical stimulus 105 according to parameters defined by the electrical biasing signal parameter data 400 and provides this signal to the electrical connector on header 220 for delivery to the patient via lead assembly 135 and electrode assembly 225.

The neurostimulator 205 may further comprise a random data generator 425 that may randomly and/or periodically generate values and/or ranges for the electrical biasing parameter data 400. The random and/or periodic values and/or ranges may be used to provide a varying electrical noise shape, such as the Gaussian, zero-mean, pseudo-random noise, and/or to randomize any other parameter as discussed above, according to a bias stimulus signal defined by the stimulation controller 420. For pseudo-random noise, a portion of the varying electrical noise shape is random and the remaining portion is dependent upon the portion which is random.

Based upon the electrical biasing parameter data 400 relating to the type of nerve stimulation to be corrected or clarified, the stimulation controller 420 provides control signals for selecting a particular type of the electrical stimulus 105 to be delivered by the neurostimulator 205 for biasing the intrinsic neural signal 110. The random data generator 425 is capable of generating randomization data that may be used to generate a number of electrical noise waveforms, such as a randomized noise signal, for use as the electrical biasing signal. The randomized noise signal may comprise various random noise types, such as Gaussian, zero-mean, or pseudo-random noise. Particular noise types may be used for various reasons, such as targeting particular nerve fibers, performing pre-polarization, or hyper-polarization, and the like. By selecting a specific noise type, various attributes, such as the current magnitude or the pulse width may be adjusted.

The random data generator 425 preferably comprises timing devices and other electronic circuitry for generating the randomization data. The random data generator 425 is also capable of generating electrical biasing signal randomization data for use in defining an electrical biasing signal comprising a non-continuous pulsed electrical signal defined by a plurality of parameters comprising a controlled (i.e., constant and/or non-random) current magnitude, a controlled pulse width, a controlled pulse period, a random on-time that varies within a first defined range, and a random off-time that varies within a second defined range. In other embodiments, one or more of the current magnitude and pulse width may be randomized with the on-time and/or off-time. In another embodiment, the random data generator 425 is capable of generating randomization data for use in defining an electrical biasing signal comprising a continuous pulsed electrical signal defined by a plurality of parameters comprising at least one of a controlled current magnitude and pulse width, and a random pulse period that varies within a defined range. In other embodiments, either or both of the current magnitude and pulse width may also be randomized. In a still further embodiment, the random data generator 425 is capable of generating randomization data for use in defining an electrical biasing signal comprising a continuous pulsed electrical signal defined by a plurality of parameters comprising at least one of a current magnitude (which may be randomized or controlled), a pulse width (which may be randomized or controlled), a polarity (which may be randomized or controlled) and, optionally, a controlled or randomized on-time and a controlled or randomized off-time.

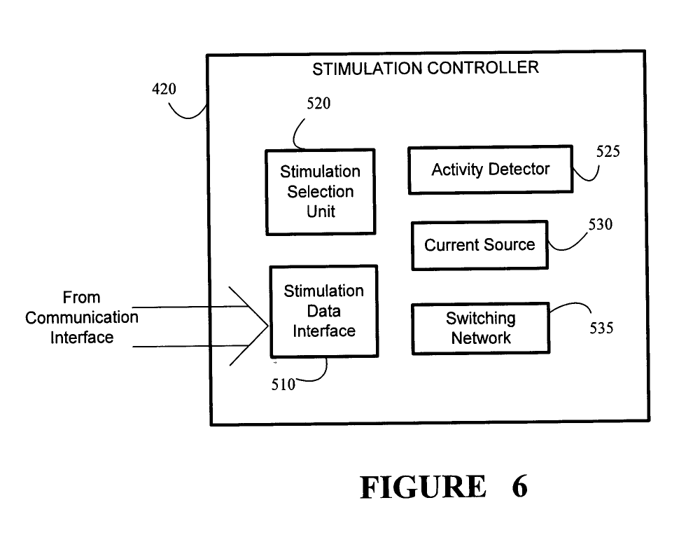

Turning now to FIG. 6, a stimulation controller 420 suitable for use in an embodiment of the present invention is provided. The controller 420 includes a stimulation data interface 510 and a stimulation selection unit 520, according to one illustrative embodiment of the present invention. The stimulation data interface 510 may receive data defining the nerve stimulation pulses, and the stimulation selection unit 520 may be capable of selecting a type of nerve stimulation to be performed by the stimulation controller 420. Examples of the types of nerve stimulation include random (including randomization of any one or more parameter), pseudo-random, and periodically random (i.e., alternating periods in which the signal is randomized for a randomization period and then non-randomized for a non-randomization period).

The stimulation data interface 510 may interface with various other portions of the implantable medical device 100, which in one embodiment comprises neurostimulator 205. For example, the stimulation data interface 510 may interface with the communication unit 170 (FIG. 1) to receive patient data from the external programming user interface 202 for programming a particular type of nerve stimulation to be performed.

In one embodiment, the stimulation data interface 510 may additionally receive data from the electrical biasing parameter data 400, which may provide parameters relating to the type of bias stimulus to be applied to the intrinsic neural signal 110. The stimulation data interface 510 may provide data to the stimulation selection unit 520, which then selects a particular type of nerve stimulation to be delivered by the neurostimulator 205. For example, the stimulation selection unit 520 may either manually or according to a program select the type of nerve stimulation via the external programming user interface 202 and the bias routine 405 (FIG. 5).

Consistent with one embodiment, the stimulation selection unit 520 may be a hardware unit comprising a processor capable of executing a program code. In an alternative embodiment, the stimulation selection unit 520 may be a software unit, a firmware unit, or a combination of hardware, software, and/or firmware. The stimulation selection unit 520 may receive data from the external programming user interface 202, via stimulation data interface 510, prompting the unit 520 to select a particular electrical biasing signal 302 (FIG. 3) for delivery by the neurostimulator 205.

In one embodiment, the electrical biasing parameter data 400 may include sensed body parameters or signals indicative of the sensed parameters, and the bias routine 405 may comprise software and/or firmware instructions to analyze the sensed electrical neural activity for determining whether electrical neurostimulation is desired. If the bias routine 405 determines that electrical neurostimulation is desired, then the neurostimulator 205 may provide an appropriate electrical biasing signal 302 to a neural structure, such as the vagus nerve 235.

The stimulation controller 420 may, in certain embodiments, further comprise an activity detector 525, although in embodiments providing purely passive stimulation it may not be present. The activity detector 525 may detect the patient parameters or signals indicative of the sensed parameters to derive electrical neural activity data for determining whether electrical neurostimulation is desired. The detected patient parameters may provide an indication of a medical condition or an indication of an event.

Using a sensing electrode pair, for example, the activity detector 525 may measure voltage fluctuations on the vagus nerve 235 to detect action potentials during an epileptic seizure. If the activity detector 525 determines that electrical neurostimulation is desired, then the activity detector 525 causes to the bias routine 405, in conjunction with the stimulus generator 150, to generate and apply the electrical biasing signal 302 to the intrinsic neural signal 110 on the vagus nerve 235. The activity detector 525 may also cause the stimulation controller 420 to switch between various electrodes employed by the neurostimulator 205 based on the detected intrinsic neural signal 110 on the vagus nerve 235. It will be recognized that one or more of the blocks 405-425 (which may also be referred to as modules) may comprise hardware, firmware, software units, or any combination thereof.

The stimulation controller 420 also comprises a current source 530 to provide a controlled current signal for delivery of the electrical biasing signal 302 to the patient. The current source 530, in one embodiment, is capable of providing a controlled current even if the impedance across the leads varies (as described below), thereby delivering the electrical biasing signal 302 from the neurostimulator 205 to a neural structure such as vagus nerve 235. Additionally, the stimulation controller 420 may comprise a switching network 535 capable of switching through various polarities and wires. For example, the switching network 535 may switch between various electrodes, i.e., the electrode(s) 140(1-n) that may be driven by the neurostimulator 205. Thus, using particular sub-modules of the stimulation controller 420 (e.g., sub-modules 510-535), the neurostimulator 205 is able to deliver electrical biasing signals in various noise shapes, durations, and polarities, and adjust the nerve stimulation in multiple electrodes of the electrode(s) 140(1-n) in various combinations.

In certain embodiments, the implantable medical device 100 may comprise a neurostimulator 205 having a case 215 as a main body in which the electronics described in FIGS. 1-5 may be enclosed and hermetically sealed. Coupled to the main body may be a header 220 designed with terminal connectors for connecting to a proximal end of the electrically conductive lead(s) 135. The main body may comprise a titanium shell, and the header may comprise a clear acrylic or other hard, biocompatible polymer such as polycarbonate, or any biocompatible material suitable for implantation into a human body. The lead(s) 135 projecting from the electrically conductive lead assembly 230 of the header may be coupled at a distal end to electrodes 140(1-n), which are coupled to neural structure such as vagus nerve 235, utilizing a variety of methods for attaching the lead(s) 135 to the tissue of the vagus nerve 235. Therefore, the current flow may take place from one terminal of the lead 135 to an electrode such as electrode 226 (FIG. 2) through the tissue, e.g., vagus nerve 235, to a second electrode such as electrode 228 and a second terminal of the lead 135.

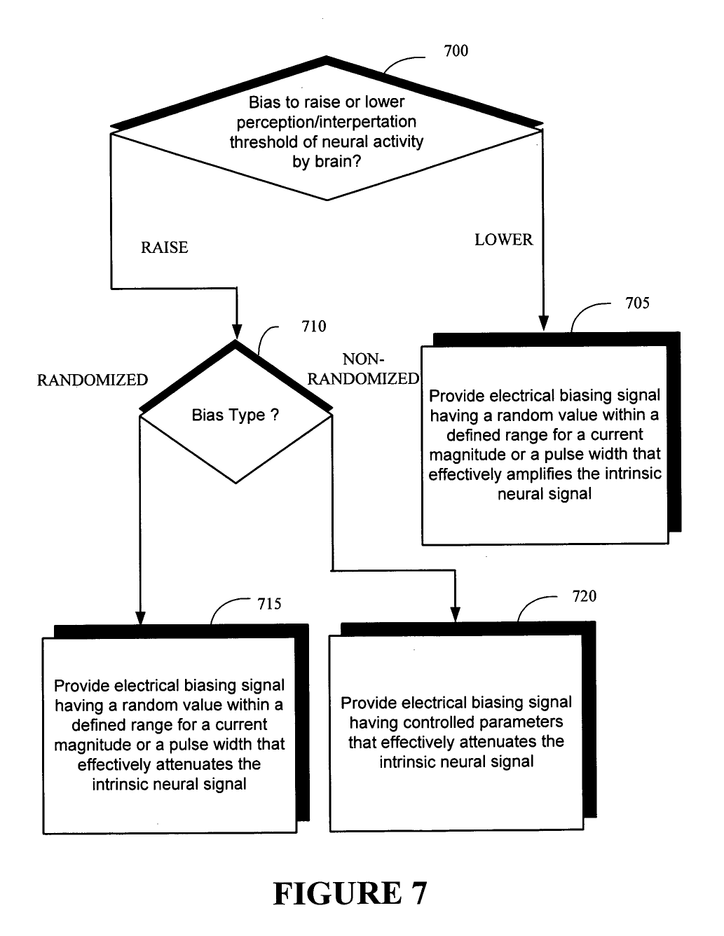

Referring to FIG. 7, a flow chart illustrates the steps of a method for biasing an intrinsic neural signal 110 in a neural structure such as vagus nerve 235 to enable or improve interpretation by the brain 115 of the patient in accordance with one illustrative embodiment of the present invention. Initially, a decision must be made whether to provide a signal to raise the interpretation threshold of the patient or to lower the threshold (block 700). The neurostimulator 205 may provide an electrical biasing signal defined so as to raise the overall level of the intrinsic neural signal 110, or to lower it (i.e., adjust a threshold of interpretation of neural activity by the brain 115 and thus alter its interpretation threshold). To this end, the neurostimulator 205 may be used to generate a randomized electrical biasing signal, a controlled electrical biasing signal, or both randomized and controlled electrical biasing signals.

Where it is desirable to lower an interpretation threshold, an electrical biasing signal 302 may be defined and applied to the neural structure so as to lower the interpretation threshold by effectively amplifying the intrinsic neural signal 110 (Block 705). The stimulus generator 150 may provide an electrical biasing signal 302 having one or more randomized parameters whose value varies within a defined range, e.g., a randomized current magnitude, pulse width, pulse period, or a pulse polarity, that effectively amplifies the intrinsic neural signal 110. The stimulus generator 150 may apply the electrical biasing signal 302 to the neural structure continuously, periodically or intermittently.

On the other hand, where it is desirable to raise the interpretation threshold, the intrinsic neural signal 110 may be biased by an electrical biasing signal 302 so as to attenuate the overall level of the neural signal. In this embodiment, the implantable medical device 100 adds an electrical biasing signal intended to allow the brain to adapt and “tune out” the signal, thereby raising the brain's interpretation threshold for the intrinsic neural signal. This may be done by providing either randomized or non-randomized signals, and a decision as to what type of signal should be applied is made (Block 710).

One way to accomplish this is by providing a controlled, non-randomized electrical biasing signal (Block 720). Non-continuous controlled electrical biasing signals are known in the art, e.g., as conventional vagus nerve stimulation. However, in certain embodiments the invention may comprise providing a continuous controlled signal, i.e., a non-random signal having no defined on-time and off-time, to the neural structure. Without being bound by theory, avoiding discrete on-times and off-times may be more effective that providing them in teaching the brain to disregard a certain portion of the intrinsic neural signal, thereby raising the threshold of activity required for the brain to interpret the intrinsic neural signal 110. However, the brain's ability to adapt to such a signal may limited or impaired because of a variety of factors, including poor electrode/nerve coupling, neural damage to either the structure being stimulated or one or more brain structures, medications, and other factors.

Accordingly, embodiments of the present invention for raising the interpretation threshold may use randomization of one or more signal to present to the brain a signal that appears to be larger and/or more controlled than existing non-randomized neurostimulation regimes (Block 715). In one such embodiment, a signal parameter may be randomized within a tightly controlled interval, for example, the electrical biasing signal 302 may comprise a pulsed, non-continuous signal in which the current magnitude is randomized from 1.0 milliamps to 1.25 milliamps, but with a controlled pulse width, pulse period, and on-time and off-time. Such a signal may be seen by the brain as a more controlled, rather than less controlled, signal because the randomization may recruit a wider variety of neural axons than a simple 1.0 milliamp signal. Without being bound by theory, this may be possible in part because such limited randomization schemes may serve to minimize side effects, such as pain, and thus allow the patient to tolerate a more powerful signal than previously employed, which is more perceptible to the brain as an essentially constant signal, and thus triggers an adaptive response, raising the interpretation threshold. Alternatively, if the intrinsic neural signal remains above an inactivity level, the addition of the randomized bias signal may allow the signal to cross below the inactivity level and allow appropriate interpretation of the inactivity.

In alternative embodiments, the electrical biasing signal may comprise both random and non-random signals. For example, where a non-continuous pulsed signal is used, a pulse burst having one or more randomized parameters, e.g., current, pulse width, and/or frequency, may be provided to the nerve for a first on-time, followed by a controlled or random off-time, and a non-random pulse burst may then be provided and applied to the nerve for second on-time, followed by alternating random and non-random pulse bursts. Pseudo-random variations in any stimulus parameter (including continuous pseudorandom stimulation) may also be employed.

In a further alternative embodiment, the electrical stimulus 105 may be applied so that a portion (between 0 and 100%) of intrinsic vagal activity in afferent neural pathways or nerve fibers may be inhibited from propagation, i.e., blocked, thereby attenuating the intrinsic neural signal 110. The electrical stimulus 105 may also be used to decelerate action potentials using sub-threshold anodic currents. In these alternate approaches, the electrical biasing may be sub-threshold (i.e., below the level required to generate action potentials on the vagus nerve 235) to block conduction of a portion of the neural traffic to clarify the overall information content.

Whether raising or lowering the interpretation threshold of the intrinsic neural signal 110, the electrical biasing signal 302 of the electrical stimulus 105 may be applied to the neural structure either passively or actively. The determination of whether to employ sensors to actively trigger stimulation or to use purely passive stimulation may be based on a potential power cost or varying efficacy based upon the condition and treatment. While generating an action potential for an individual nerve fiber is generally an “all-or-nothing,” threshold-based phenomena, the electrical biasing signal 302 of the present invention may, when the thousands of fibers within the nerve 120 such as the vagus nerve 235 are considered, provide an adjustment over a wide continuum. To produce a desired level of neurons firing in the nerve 120 or a nerve trunk, resulting in an improved interpretation of the collective (i.e., biased) signal by the brain, the nerve stimulation may take advantage of temporal or spatial summation in a nerve bundle.

Advantageously, neurostimulators 205 according to the present invention may provide an electrical biasing signal that is sufficient to clarify the existing or intrinsic vagal activity, even reducing stimulation intensity in some situations. Since the improved nerve stimulation may be stochastic rather than patterned, the neurostimulator 205 according to the present invention may also eliminate some VNS side effects. While sensing or detection of the existing or intrinsic vagal activity may be employed to determine the electrical stimulus 105, in one embodiment of present invention, it is to be understood that, such sensing or detection of the existing or intrinsic vagal activity should not be used to limit the scope of the instant invention.

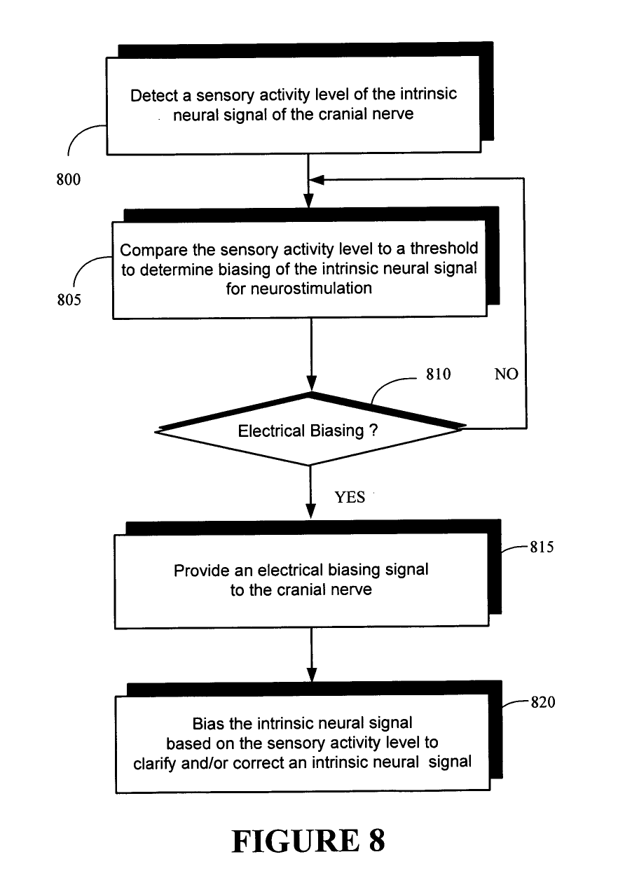

Referring to FIG. 8, a flow chart representation is provided of the steps for applying the electrical stimulus 105, such as an electrical biasing signal 302, to a neural structure such as the vagus nerve 235, in accordance with one illustrative embodiment of the present invention. Application of the electrical stimulus 105 may enable the brain 115 of the patient to interpret previously undecipherable electrical signals in the intrinsic neural signal 110, thereby providing treatment to a patient having a neurologically mediated disease, condition or disorder. Referring to FIG. 6, an activity detector 525 in a neurostimulator 205 may detect an activity level of the intrinsic neural signal 110 in a neural structure such as vagus nerve 235, being provided to the brain 115 of the patient (block 800). The bias routine 405 may compare the activity level to a threshold, such as a pain threshold stored in the electrical biasing parameter data 400 to determine biasing of the intrinsic neural signal 110 for interpretation (block 805).

A determination may be made by the bias routine 405 for the neurostimulator 205 to ascertain whether an electrical biasing signal is desired for clarifying or correcting the intrinsic neural signal to enable or improve interpretation thereof by the brain 115 (decision block 810). If a need for an adjustment of the nerve stimulation is indicated, the stimulus generator 150 may provide the electrical biasing signal 302 to the neural structure, such as the cranial nerve 120 (block 815). The delivery of the electrical biasing signal 302 may bias the intrinsic neural signal 110 based on a sensed intrinsic neural signal activity level to clarify and/or correct the intrinsic neural signal 110 (block 820). Conversely, the bias routine 405 continues to check whether the electrical stimulus 105 is desired (decision block 810).

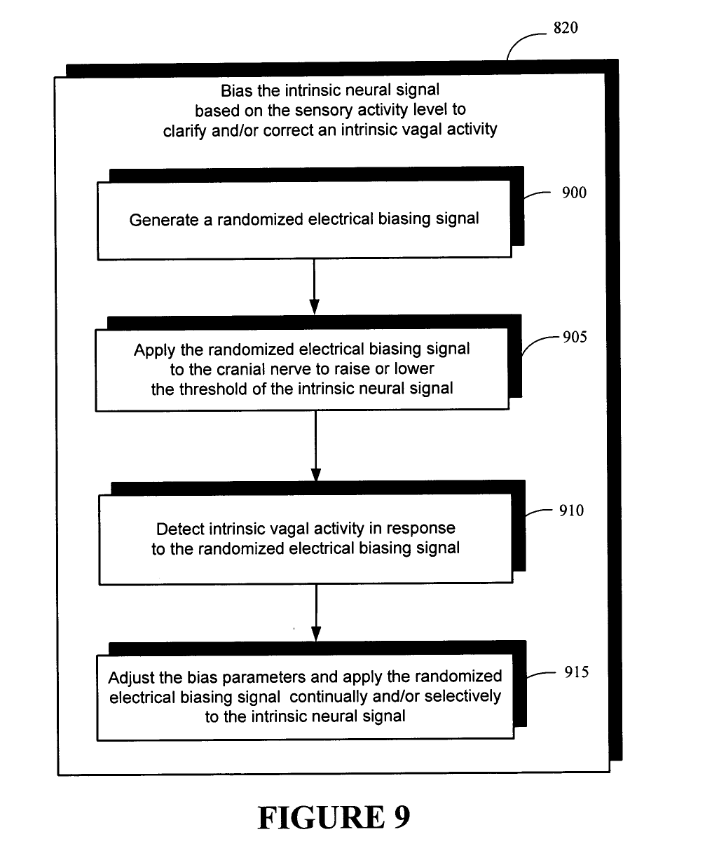

Referring to FIG. 9, a flow chart illustrates the steps of biasing the intrinsic neural signal 110 from a nerve such as vagus nerve 235 to enable or improve interpretation by the brain 115 of the patient in accordance with one illustrative embodiment of the present invention. To this end, the stimulus generator 150 may generate a randomized electrical biasing signal (block 900). To raise or lower the overall level of the intrinsic neural signal 110 (and thus alter its interpretation threshold by the brain, the electrical biasing signal 302 may be applied to the vagus nerve 235 which, without being bound by theory, may generate a multiplicity of action potentials in the vagus nerve (block 905). The activity detector 525 may detect the biased intrinsic vagal activity using one or more of the electrode(s) 140(1-n) as sensors (block 910). The stimulus generator 150 may adjust the electrical biasing parameters and apply the randomized electrical biasing signal continuously, periodically or intermittently to the intrinsic neural signal 110 (block 915).

The intrinsic neural signal 110 may be biased by lowering the interpretation threshold or effectively raising (i.e., amplifying) the information level of the neural signal. For example, the implantable medical device 100 such as neurostimulator 205 may add noise to the intrinsic neural signal of the vagus nerve 235, which results in an effectively lowered threshold for the brain to interpret the intrinsic vagal activity. In another embodiment, the intrinsic neural signal 110 may be biased by attenuating (i.e., lowering) the overall level of the neural signal using the biased intrinsic vagal activity. In this embodiment, the implantable medical device 100 applies an inhibitive stimulus to subtract neural activity and raise the threshold of interpretation by reducing chatter.

In one embodiment, the electrical stimulus 105 may comprise continuous low-level stochastic stimulation, addressing power consumption concerns. Likewise, use of stochastic resonance for biasing afferent vagal neurons may result in therapy improvements in a host of diseases and/or disorders, including but not limited to movement disorders such as epilepsy and Parkinson's disease; neuropsychiatric disorders including depression, bipolar disorder, anxiety, obsessive-compulsive disorders, schizophrenia, autism, and attention deficit/hyperactivity disorder; eating disorders including bulimia, obesity and anorexia nervosa; substance addictions; sleep disorders such as chronic fatigue syndrome and narcolepsy; pain conditions such as migraines and cluster headaches; post-traumatic stress syndrome; dementia including Alzheimer's Disease; cognitive disorders including alertness, sleepiness, memory functions, critical thinking, reasoning, speech, work/educational performance, response inhibition, language skills, interpretive understanding; endocrine disorders including diabetes; digestive disorders including hypermotility, hypomotility, Crohn's Disease, colitis; traumatic brain injury; degenerative diseases; learning disabilities; motor and coordination diseases; cardiac conditions; immune system deficiencies; pulmonary and respiratory disorders; and all disorders impacted by or related to the autonomic nervous system.

In one embodiment, neurostimulators 205 of the present invention may be used not only for the treatment of diseases, disorders, or medical conditions, but also for enhancement (e.g. cognitive skills) of sensory or neural function, should the benefits outweigh the costs. Furthermore, treatment of diseases may benefit from neurostimulators 205 of the present invention. The VNS therapy by the neurostimulator 205 may be applied to any of variety of nerve or nerves in human body, e.g., vagal afferent stimulation. However the electrical biasing signal 302 based therapy of the present invention may be applied to any cranial nerve. In addition, the methods and apparatus of the present invention may be applied to any part of the CNS, e.g., the spinal cord and/or brain.

The electrical biasing signals of the present invention may also be applied to any portion of the peripheral nervous system (PNS). The modes of nerve stimulation may include stochastic resonance (SR) alone, stochastic resonance with a conventional VNS (i.e., non-random signals), and stochastic resonance and other forms of conventional neurostimulation. The stochastic resonance based biasing applied to all forms of neural stimulation may be beneficial for treating patients suffering from different diseases, disorders, or cognitive skill deficiency. To this end, the neurostimulator 205 may provide various forms of bias stimulation for VNS therapy. In this manner, the neurostimulator 205 may significantly improve the treatment of diseases, disorders, or cognitive skill deficiency or provide an enhanced therapy by using a bias signal (either non-random or random) to improve the CNS interpretation of intrinsic neural information.

However, in some embodiments, to provide vagus nerve stimulation (VNS) therapy, a patient's medical condition may also be monitored using the neurostimulator 205. Sensing-type electrodes, such as the electrodes(s) 140(1-n) may implanted at or near the vagus nerve 235. Using the sensing electrodes(s) 140(1-n), the patient's medical condition may be detected and associated data may be measured against a predetermined threshold level. If the patient's medical condition exceeds the predetermined threshold level over a given period, the stimulus generator 150 may be triggered to apply a therapeutic electrical biasing signal. The therapeutic electrical biasing signal 302 may be applied periodically or applied as a result of patient intervention by manual activation of the stimulus generator 150 using external control.

Use of the neurostimulator 205 may improve efficacy of the VNS therapy in many neurological or neuropsychiatric conditions. In particular, when certain emotions result from visceral changes in the patient's body 200 and the brain's interpretation of vagal signals carrying sensory afferent information that causes affective emotions (e.g., anxiety and depression), the therapeutic electrical biasing signal 302 may provide a desired mechanism of action. Other anxiety disorders involving a faulty interpretation or erratic availability of this neural information to the brain may also be treated by methods and apparatus of the present invention involving electrical biasing signals. Accordingly, the electrical stimulus 105 may bias the intrinsic neural signal 110 in a way that provides an appropriate mechanism of action for desired nerve stimulation. In this manner, the neurostimulator 205 may improve the efficacy of VNS therapy in some neurological or neuropsychiatric medical conditions.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. A neurostimulation system for treating a patient with a medical condition comprising:
a stimulus generator to generate an electrical biasing signal for at least a target portion of a neural structure of a patient, said electrical biasing signal comprising a pulsed electrical signal defined by at least one parameter selected from the group consisting of a voltage magnitude, a current magnitude, a pulse width, a pulse period, an on-time and an off-time, and wherein at least one of said voltage magnitude, said current magnitude, said pulse width, said pulse period, said on-time and said off-time comprises a random value that varies within a defined range;
at least one electrode coupled to said stimulus generator and to a neural structure of the patient; and
a controller operatively coupled to said stimulus generator, said controller being adapted to apply said electrical biasing signal to said neural structure to bias an intrinsic neural signal on the neural structure.

2. The system of claim 1 further comprising a random data generator for generating said random value for said at least one parameter.

3. The system of claim 1 further comprising a memory for storing said defined range.

4. The system of claim 1 wherein said neural structure comprises a cranial nerve, a sympathetic nerve, a spinal cord structure, and a structure within the patient's brain.

5. The neurostimulation system of claim 1 wherein said at least one parameter of the electrical biasing signal comprises a voltage magnitude, and wherein said voltage magnitude of said pulses is random and varies within a range within the range of from −15.0 volts to 15.0 volts.

6. The neurostimulation system of claim 1 wherein said at least one parameter of the electrical biasing signal comprises a current magnitude and wherein said current magnitude of said pulses is random and varies within a range within the range of from −8.0 milliamps to 8.0 milliamps.

7. The neurostimulation system of claim 6 wherein said current magnitude of said pulses is random and varies within a range within the range of from −3.0 milliamps to 3.0 milliamps.

8. The neurostimulation system of claim 1 wherein said at least one parameter of the electrical biasing signal comprises a pulse width and wherein said pulse width of said pulses is random and varies within a range within the range of from 1 microsecond to 1 second.

9. The neurostimulation system of claim 1 wherein said at least one parameter of the electrical biasing signal comprises a current magnitude and a pulse width, and wherein said current magnitude comprises a random value that varies within a first defined range and said pulse width comprises a random value that varies within a second defined range.

10. The neurostimulation system of claim 1 wherein said at least one parameter of the electrical biasing signal comprises a pulse period and wherein said pulse period of said pulses is random and varies within a range within the range of from 1 microsecond to 1 second.

11. The neurostimulation system of claim 1 wherein said at least one parameter of the electrical biasing signal comprises an on-time and wherein said on-time is random and varies within a range within the range of from 1 second to 24 hours.

12. The neurostimulation system of claim 1 wherein said at least one parameter of the electrical biasing signal comprises an off-time and wherein said off-time is random and varies within a range within the range of from 1 second to 24 hours

13. The neurostimulation system of claim 1 further comprising a sensor for detecting an intrinsic neural signal on said neural structure.

14. The neurostimulation system of claim 13 further comprising:
a signal analysis unit for comparing said detected intrinsic neural signal to a threshold of intrinsic neural activity;
wherein said controller further comprises a switching network for applying said electrical biasing signal to said neural structure in response to said signal analysis unit.

15. The neurostimulation system of claim 14, wherein said controller further comprises a stimulation selection unit for adjusting at least one of said at least one parameters in response to said comparing step.

16. The neurostimulation system of claim 1 wherein said defined range for said at least one parameter comprises an upper limit and a lower limit, and wherein at least one of said upper limit and said lower limited is defined based upon a pain threshold of the patient.

17. The neurostimulation system of claim 1, wherein said electrical biasing signal comprises a pulsed noise signal.

18. The neurostimulation system of claim 1, wherein said at least one electrode comprises a pair of electrodes for contacting said neural structure for direct stimulation thereof.

19. The neurostimulation system of claim 1, further comprising a communication interface and a programming unit in communication with said communication interface, wherein said programming unit is capable of programming said a least one parameter defining said electrical biasing signal.

20. The neurostimulation system of claim 1, wherein said pulsed electrical signal further comprises a first time interval wherein at least one of said voltage magnitude, said current magnitude, said pulse width, said pulse period, said on-time and said off-time comprises a random value that varies within a defined range, and a second time interval wherein said at least one parameter comprising a random value in said first time interval comprises a non-random value.

21. The method of claim 1 wherein said random value varies within a defined range on a pulse-to-pulse basis.

22. The method of claim 1 wherein said random value varies within a defined range on a burst-to-burst basis.

23. A neurostimulator for providing an electrical stimulation therapy to a patient comprising:
a stimulus generator to generate an electrical biasing signal for an intrinsic neural signal in a neural structure of the patient, said electrical biasing signal comprising a pulsed electrical signal defined by a plurality of parameters comprising at least a current magnitude, a pulse width, and a pulse period, wherein said pulse period comprises a random value that varies within a defined range;
at least one electrode coupled to said stimulus generator and to said neural structure; and
a controller coupled to said stimulus generator and adapted to apply said electrical biasing signal to said neural structure of the patient.

24. The neurostimulator of claim 23 wherein said neural structure comprises a cranial nerve, a sympathetic nerve, a spinal cord structure, and a structure within the patient's brain.

25. The neurostimulator of claim 23 wherein said current magnitude comprises a constant magnitude.

26. The neurostimulator of claim 23 wherein said current magnitude comprises a random value that varies within a defined range.

27. The neurostimulator of claim 23 wherein said pulse width comprises a random value that varies within a defined range.

28. The neurostimulator of claim 23 wherein said electrical biasing signal comprises a continuous electrical signal.

29. The neurostimulator of claim 23 wherein said plurality of parameters defining said pulsed electrical signal further comprises an on-time and an off-time, and wherein said on-time and said off-time may comprise a random value or a non-random value.

30. The neurostimulator of claim 23 further comprising a sensor for detecting said intrinsic neural signal on said neural structure.

31. The neurostimulation system of claim 30 further comprising:
a signal analysis unit for comparing said detected intrinsic neural signal to a threshold of intrinsic neural activity;
wherein said controller further comprises a switching network for applying said electrical biasing signal to said neural structure in response to said signal analysis unit.

32. The neurostimulation system of claim 31, wherein said plurality of parameters defining said pulsed electrical signal further comprises an on-time and an off-time, and wherein said on-time and said off-time may comprise a random value within a defined range or a non-random value, and wherein said controller further comprises a stimulation selection unit for adjusting at least one of said defined range or said non-random value for one of said on-time or said off-time in response to said signal analysis unit.

33. A neurostimulator for providing an electrical stimulation therapy to a patient comprising:
a stimulus generator to generate an electrical biasing signal for an intrinsic neural signal in a neural structure of the patient, said electrical biasing signal comprising a pulsed electrical signal defined by a plurality of parameters comprising a constant current magnitude, a constant pulse width, an on-time and an off-time, wherein at least one of said on-time and said off-time comprises a random value that varies within a defined range;
at least one electrode coupled to said stimulus generator and to said neural structure; and
a controller coupled to said stimulus generator and adapted to apply said electrical biasing signal to said neural structure of the patient.

34. The neurostimulator of claim 30 wherein said on-time comprises a random value that varies within a first defined range, and said off-time comprises a random value that varies within a second defined range.

35. The neurostimulator of claim 30 wherein said plurality of parameters defining said pulsed electrical signal further comprises a frequency selected from the group consisting of a non-random frequency, a random frequency within a defined frequency range, or a swept frequency within a defined range.

36. The neurostimulator of claim 30 wherein said plurality of parameters defining said pulsed electrical signal further comprises a pulse period selected from the group consisting of a constant pulse period and a random pulse period that varies within a defined range.

37. A neurostimulator for providing an electrical stimulation therapy to a patient comprising:
a stimulus generator to generate an electrical biasing signal for an intrinsic neural signal in a neural structure of the patient, said electrical biasing signal comprising an electrical signal defined by a plurality of parameters comprising a current magnitude and at least one of an on-time and an off-time, wherein at least one of said current magnitude, said on-time and said off-time comprises a random value that varies within a defined range;
at least one electrode coupled to said stimulus generator and to said neural structure; and
a controller coupled to said stimulus generator and adapted to apply said electrical biasing signal to said neural structure of the patient.

38. The neurostimulator of claim 37 wherein said electrical signal comprises a non-pulsed electrical signal.

39. The neurostimulator of claim 37 wherein said electrical signal comprises a charge-balanced electrical signal.

40. The neurostimulator of claim 37 wherein said electrical biasing signal comprises a noise signal having a random current magnitude that varies within a range within the range of from −8.0 to 8.0 milliamps.

41. The neurostimulator of claim 37 wherein said on-time is random and varies within a range within the range of from 1 second to 24 hours, and wherein said off-time is random and varies within a range within the range of from 1 second to 24 hours.

42. A neurostimulator for providing an electrical stimulation therapy to a patient comprising:
a stimulus generator to generate an electrical biasing signal for an intrinsic neural signal in a neural structure of the patient, said electrical biasing signal comprising a non-pulsed, continuous electrical signal defined by at least a current magnitude, wherein said current magnitude is random and varies within a range within the range of from −8.0 to 8.0 milliamps;
at least one electrode coupled to said stimulus generator and to said neural structure; and
a controller coupled to said stimulus generator and adapted to apply said electrical biasing signal to said neural structure of the patient.

43. A neurostimulator for providing an electrical stimulation therapy to a patient comprising:
a stimulus generator to generate an electrical biasing signal comprising an electrical noise signal for biasing an intrinsic neural signal in a neural structure selected from the group consisting of a cranial nerve, a brain structure, a spinal cord structure, and a sympathetic nerve structure;
at least one electrode coupled to said stimulus generator and to said neural structure; and
a controller coupled to said stimulus generator and adapted to apply said electrical biasing signal to said neural structure of the patient.

44. The neurostimulator of claim 43 wherein said electrical noise signal comprises a noise signal selected from the group consisting of a zero-mean, pseudo-random, or Gaussian noise signal.
